(19) United States
(12) Patent Application Publication
(10) Pub. No.: US 2009/0239929 A1
(43) Pub. Date: Sep. 24, 2009

(54) N-Pyrrolidin-3-YL-Amide Derivatives As Serotonin and Noradrenalin Re-Uptake Inhibitors

(76) Inventors: Paul Vincent Fish, Sandwich (GB); Thomas Ryckmans, Sandwich (GB); Alan Stobie, Sandwich (GB); Florian Wakenhut, Sandwich (GB)

Correspondence Address: PFIZER INC.;PATENT DEPARTMENT, Bld 114 M/S 114, EASTERN POINT ROAD, GROTON, CT, 06340, US

(21) Appl. No.: 11/721,359

(22) PCT Filed: Dec. 2, 2005
(86) PCT No.: PCT/IB05/03742

§ 371 Date: Aug. 3, 2007

Related U.S. Application Data
(60) Provisional application No. 60/648,230, filed on Jan. 28, 2005.
(60) Provisional application No. 60/695,216, filed on Jun. 28, 2005.

(30) Foreign Application Priority Data
Dec. 14, 2004 (GB) 0427383.5

(51) Int. Cl. A61K 31/40; C07D 207/14; A61P 13/00; A61P 25/24; A61P 25/00; A61P 15/12

(52) U.S. Cl. 514/426; 548/557

(57) ABSTRACT

A compound of Formula (I) and pharmaceutically and/or veterinarily acceptable derivatives thereof, wherein: R¹ is H, C₁₋₆alkyl, —C(A)D, C₃₋₈cycloalkyl, aryl, het, aryl-C₁₋₄alkyl or het-C₁₋₄alkyl, wherein the cycloalkyl, aryl or het groups are optionally substituted; A is S or O; D is H, C₁₋₆alkyl, aryl, het, aryl-C₁₋₄alkyl or het-C₁₋₄alkyl; aryl represents phenyl, naphthyl, anthracyl or phenanthryl; het represents an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom; R² is aryl¹ or het¹, each optionally substituted; n is 1 or 2, provided that when n is 1, m is 0 or 1 and when n is 2, m is 0, wherein if m is 0, then * represents a chiral centre; R³ is (CH₂)ₐE, wherein a is 0, 1 or 2 and E is a group selected from: Formula (i) wherein: X is O, S, NR¹², (CH₂)ᵥ or a bond; b is 1, 2, 3 or 4; c is 1, 2 or 3; v is 1 or 2; R¹⁰ and ¹¹ are each independently H or C₁₋₄ alkyl; and R¹² is H, ₁₋₆ alkyl, C(O)C₁₋₆ alkyl, SO₂—C₁₋₆alkyl; and wherein one or more pairs of hydrogen atoms on adjacent carbon or nitrogen atoms may be replaced by a corresponding number of double bonds, provided the ring system is not aromatic; Formula (ii) a carbocyclic spiro group containing 6 to 12 carbon atoms; Formula (iii) wherein: d is 1, 2, 3 or 4; a is 1, 2 or 3; f is 1 or 2; and R³⁰ is H or C₁₋₄alkyl; and wherein one or more pairs of hydrogen atoms on adjacent carbon atoms may be replaced by a corresponding number of double bonds, provided the ring system is not aromatic; Formula (iv) wherein: g is 0, 1, 2 or 3; J is NR⁴⁰; and R⁴⁰ is C(O)C₁₋₆ alkyl, S0₂-C₁₋₆alkyl; Formula (v) wherein: h is 0, 1, 2 or 3; and R⁵⁰ is H, C₁₋₈alkyl, C₁₋₈alkoxy, OH, halo, CF₃, OCF₃, SCF₃, hydroxy-C₁₋₆alkyl, C₁₋₄alkoxy-C₁₋₆alkyl and C₁₋₄alkyl-S—C₁₋₄alkyl; Formula (vi) —CH(cyclopropane)₂; Formula (vii) C₁₋₆alkyl, substituted by at least one substituent; and Formula (viii) C₃₋₈cycloalkyl-C₁₋₆alkyl; wherein the C₁₋₆alkyl moiety is substituted at any point other than at the junction with the C₃₋₈cycloalkyl moiety, by at least one substituent. The compounds exhibit activity as both serotonin and/or noradrenaline re-uptake inhibitors and therefore have utility in a variety of therapeutic areas, for example urinary incontinence.

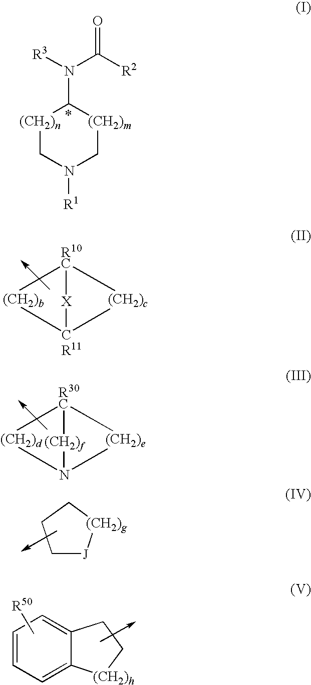

This invention relates to novel amide compounds which inhibit monoamine re-uptake, to processes for their preparation, to pharmaceutical compositions containing them and to their use in medicine.

The compounds of the invention exhibit activity as serotonin and/or noradrenaline re-uptake inhibitors and therefore have utility in a variety of therapeutic areas. For example, the compounds of the invention are of use in the treatment of disorders in which the regulation of monoamine transporter function is implicated, more particularly disorders in which inhibition of re-uptake of serotonin or noradrenaline is implicated. Furthermore, the compounds of the invention are of use in disorders in which inhibition of both serotonin and noradrenaline is implicated, such as urinary incontinence. Additionally, the compounds of the invention are of use in disorders in which it may be desired to inhibit preferentially the reuptake of one of noradrenaline or serotonin compared with the other, such as pain.

According to a first aspect, the invention provides a compound of formula (I),

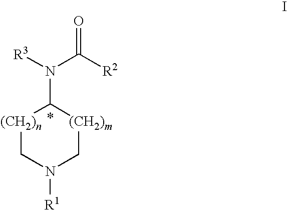

and pharmaceutically and/or veterinarily acceptable derivatives thereof, wherein:

R¹ is H, C₁₋₆alkyl, —C(A)D, C₃₋₈cycloalkyl, aryl, het, aryl-C₁₋₄alkyl or het-C₁₋₄alkyl, wherein the cycloalkyl, aryl or het groups are optionally substituted by at least one substituent independently selected from C₁₋₈alkyl, C₁₋₈alkoxy, OH, halo, CF₃, OCHF₂, OCF₃, SCF₃, hydroxy-C₁₋₆alkyl, C₁₋₄alkoxy-C₁₋₆alkyl and C₁₋₄alkyl-S—C₁₋₄alkyl;
A is S or O;
D is H, C₁₋₆alkyl, aryl, het, aryl-C₁₋₄alkyl or het-C₁₋₄alkyl;
aryl represents phenyl, naphthyl, anthracyl or phenanthryl;
het represents an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom;

R² is aryl¹ or het¹, each optionally substituted by at least one substituent independently selected from B;
B represents C₁₋₈alkyl, C₁₋₈alkoxy, aryl², het², Oaryl², Ohet², SC₁₋₆alkyl, Saryl², Shet², OH, halo, CF₃, CHF₂, OCHF₂, OCF₃, SCF₃, CF₂CF₃, CH₂CF₃, CF₂CH₃, hydroxy-C₁₋₆alkyl, C₃₋₆cycloalkyl, C₃₋₆cycloalkyl-C₁₋₄alkyl, C₃₋₆cycloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl, C₁₋₄alkoxy-C₁₋₆alkyl, aryl²-C₁₋₄alkyl and C₁₋₄alkyl-S—C₁₋₄alkyl, wherein the aryl² and het² groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃;
aryl¹ represents phenyl, naphthyl, anthracyl or phenanthryl;
het¹ is an aromatic 5- or 6-membered heterocycle which contains at least one N, O or S heteroatom, optionally fused to an aryl group;
at each occurrence aryl² independently represents phenyl, naphthyl, anthracyl or phenanthryl;
at each occurrence het² independently represents an aromatic or non-aromatic 4-, 5- or 6-membered heterocycle which contains at least one N, O or S heteroatom, optionally fused to a 5- or 6-membered carbocyclic group or a second 4-, 5 or 6-membered heterocycle which contains at least one N, O or S heteroatom;

n is 1 or 2, provided that when n is 1, m is 0 or 1 and when n is 2, m is 0, wherein if m is 0, then * represents a chiral centre;

R³ is (CH₂)ₐE, wherein a is 0, 1 or 2 and E is a group selected from:

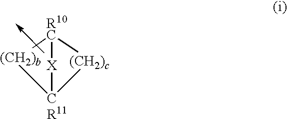

wherein:
X is O, S, NR¹², (CH₂), or a bond;
b is 1, 2, 3 or 4;
c is 1, 2 or 3;
v is 1 or 2;
R¹⁰ and Rot are each independently H or C₁₋₄alkyl; and
R¹² is H, C₁₋₄ alkyl, C(O)C₁₋₆alkyl, SO₂—C₁₋₆alkyl;
and wherein one or more pairs of hydrogen atoms on adjacent carbon or nitrogen atoms may be replaced by a corresponding number of double bonds, provided the ring system is not aromatic;

(ii) a carbocyclic spiro group containing 6 to 12 carbon atoms;

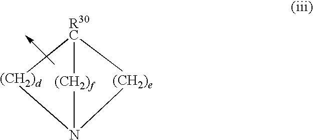

wherein:
d is 1, 2, 3 or 4;
e is 1, 2 or 3;
f is 1 or 2; and
R³⁰ is H or C₁₋₁₄alkyl;
and wherein one or more pairs of hydrogen atoms on adjacent carbon atoms may be replaced by a corresponding number of double bonds, provided the ring system is not aromatic;

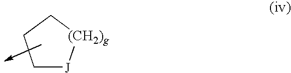

wherein:
g is 0, 1, 2 or 3;
J is NR⁴⁰; and
R⁴⁰ is C(O)C₁₋₆alkyl, SO₂—C₁₋₆alkyl;

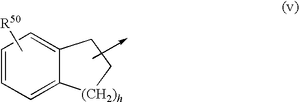

wherein:
h is 0, 1, 2 or 3; and
R⁵⁰ is H, C₁₋₄alkyl, C₁₋₄alkoxy, OH, halo, CF₃, OCF₃, SCF₃, hydroxy-C₁₋₆alkyl, C₁₋₄alkoxy-C₁₋₄alkyl and C₁₋₄alkyl-S—C₁₋₄alkyl;
(vi) —CH(cyclopropane)₂;
(vii) C₁₋₆alkyl, substituted by at least one substituent independently selected from C₁₋₆alkoxy, OH, halo, CF₃, OCF₃, SCF₃, CN, CF₂CF₃, CF₂—C₁₋₄alkyl, hydroxy-C₁₋₄alkyl, C₁₋₄alkoxy-C₁₋₆alkyl and C₁₋₄alkyl-S—C₁₋₄alkyl; and
(viii) C₃₋₈cycloalkyl-C₁₋₆alkyl; wherein the C₁₋₆alkyl moiety is substituted at any point other than at the junction with the C₃₋₈cycloalkyl moiety, by at least one substituent independently selected from C₁₋₆alkoxy, OH, halo, CF₃, OCF₃, SCF₃, CN, CF₂CF₃, CF₂—C₁₋₄alkyl, hydroxy-C₁₋₆alkyl, C₁₋₄alkoxy-C₁₋₄alkyl and C₁₋₄alkyl-S—C₁₋₄alkyl; and the C₃₋₈cycloalkyl moiety is optionally substituted by at least one substituent independently selected from C₁₋₈alkyl, C₁₋₆alkoxy, OH, halo, CF₃, OCF₃, SCF₃, CN, CF₂CF₃, CF₂—C₁₋₄alkyl, hydroxy-C₁₋₆alkyl, C₁₋₄alkoxy-C₁₋₆alkyl and C₁₋₄alkyl-S—C₁₋₄alkyl.

In an embodiment of the invention, R¹ is H.

In a further embodiment of the invention, m is 0. When m is 0, * represents the R or S enantiomeric configuration. Thus, in a further embodiment, m is 0, and * represents the S enantiomer. In a yet further embodiment n is 1.

In a still further embodiment aryl¹ represents phenyl and napthyl; more preferably aryl¹ represents phenyl. In a still further embodiment aryl² represents phenyl.

In a further embodiment het¹ represents pyridinyl or quinolinyl, optionally substituted by at least one substituent independently selected from B; preferably het¹ represents pyridinyl.

In a still further embodiment, R² is aryl¹ optionally substituted by at least one substituent independently selected from B.

In a further embodiment B represents C₁₋₈alkyl, C₁₋₈alkoxy, aryl², Oaryl², SC₁₋₆alkyl, Saryl², C₃₋₆cycloalkyl, halo, CF₃, OCF₃, OCHF₂, CF₂CH₃, C₃₋₆cycloalkyl-C₁₋₄alkyloxy, and aryl²-C₁₋₄alkyl, wherein the aryl² group is independently optionally substituted by at least one group selected from C₁₋₆alkyl, C₃₋₄cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃; preferably the aryl² group is independently optionally substituted by at least one group selected from halo; preferably halo represents chloro and fluoro; more preferably halo represents fluoro.

In a further embodiment B represents C₁₋₈alkyl, C₁₋₈alkoxy, Oaryl², SC₁₋₆alkyl, C₃₋₆cycloalkyl, halo, CF₃, OCF₃, wherein the aryl² group is independently optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃; preferably the aryl² group is independently optionally substituted by at least one group selected from halo; preferably C₁₋₈alkyl or C₁₋₈alkyloxy groups represent C₁₋₆alkyl or C₁₋₆alkyloxy, more preferably they represent C₁₋₄alkyl or C₁₋₄alkyloxy, even more preferably they represent C₁₋₂alkyl or C₁₋₂alkyloxy; preferably halo represents chloro and fluoro; more preferably halo represents fluoro.

In a further embodiment B represents C₁₋₄alkoxy, Oaryl², SC₁₋₆alkyl, halo, CF₃, wherein the aryl² group is independently optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃; preferably the aryl² group is independently optionally substituted by at least one group selected from halo; preferably C₁₋₈alkyloxy represents C₁₋₆alkyloxy, more preferably it represents C₁₋₄alkyloxy, even more preferably it represents C₁₋₂alkyloxy; preferably C₁₋₆alkyl represents C₁₋₄alkyl, more preferably it represents C₁₋₂alkyl; preferably halo represents chloro and fluoro; more preferably halo represents fluoro.

In a further embodiment B represents C₁₋₈alkoxy, Oaryl², halo, CF₃, wherein the aryl² group is independently optionally substituted by at least one group selected from C₁₋₆alkyl, C₃₋₆cycloalkyl, C₁₋₆alkoxy, OC₃₋₆-cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃; preferably the aryl² group is independently optionally substituted by at least one group selected from halo; preferably C₁₋₈alkyloxy represents C₁₋₆alkyloxy, more preferably it represents C₁₋₄alkyloxy, even more preferably it represents C₁₋₂alkyloxy; preferably C₁₋₆alkyl represents C₁₋₄alkyl, more preferably it represents C₁₋₂alkyl; preferably halo represents chloro and fluoro; more preferably halo represents fluoro.

In a still further embodiment, aryl¹ and het¹ are independently substituted by one, two or three substituents as defined above. More particularly aryl¹ and het¹ are substituted by one or two substituents as defined above.

In a still further embodiment, aryl² and het² are independently substituted by one, two or three substituents as defined above. More particularly aryl² and het² are substituted by one or two substituents as defined above.

In a further embodiment E is selected from (i), (ii), (vi), and (vii) as described above; preferably E is selected from (i), and (vii) as described above. In a still further embodiment, a represents 0 or 1; preferably a represents 0. In a further embodiment, b represents 2 or 3. In a still further embodiment c represents 2. In a still further embodiment, X represents (CH₂)ᵥ or a bond. In a yet still further embodiment v represents 1. In a further embodiment R¹⁰ represents H. In a still further embodiment, R¹¹ represents H.

In a further embodiment, (vii) represents C₁₋₆alkyl, substituted by at least one substituent independently selected from C₁₋₆alkyloxy, halo and CF₃; preferably C₁₋₆alkyloxy is methoxy; preferably halo is selected from chloro or fluoro; more preferably halo is fluoro. In a further embodiment, (vii) represents C₁₋₃alkyl, substituted by at least one substituent independently selected from C₁₋₆alkyloxy, halo and CF₃; preferably C₁₋₆alkyloxy is methoxy; preferably halo is selected from chloro or fluoro; more preferably halo is fluoro.

In an alternative embodiment, there is provided a compound of formula I′:

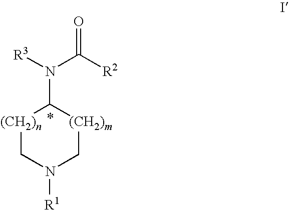

and pharmaceutically and/or veterinarily acceptable derivatives thereof, wherein:

R¹ is H, C₁₋₆alkyl, —C(A)D, C₃₋₈cycloalkyl, aryl, het, aryl-C₁₋₄alkyl or het-C₁₋₄alkyl, wherein the cycloalkyl, aryl or het groups are optionally substituted by at least one substituent independently selected from C₁₋₈allyl, C₁₋₈alkoxy, OH, halo, CF₃, OCHF₂, OCF₃, SCF₃, hydroxy-C₁₋₆alkyl, C₁₋₄alkoxy-C₁₋₆alkyl and C₁₋₄alkyl-S—C₁₋₄alkyl;

R² is aryl or heteroaryl, each optionally substituted by at least one substituent independently selected from C₁₋₄alkyl, C₁₋₈alkoxy, aryl¹, het¹, Oaryl¹, Ohet¹, Saryl¹, Shet¹, OH; halo, CF₃, CHF₂, OCHF₂, OCF₃, SCF₃, CF₂CF₃, CH₂CF₃, CF₂CH₃, hydroxy-C₁₋₄alkyl, C₃₋₆cycloalkyl, C₃₋₆cycloalkyl-C₁₋₄alkyl, C₃₋₆cyloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl, C₁₋₄alkoxy-C₁₋₆alkyl and C₁₋₄alkyl-S—C₁₋₄alkyl, wherein the aryl¹ and het¹ groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₆alkyl, SC₁₋₆alkyl and SCF₃;

A is S or O;

D is H, C₁₋₄alkyl, aryl, het, aryl-C₁₋₄alkyl or het-C₁₋₄alkyl;

n is 1 or 2, provided that when n is 1, m is 0 or 1 and when n is 2, m is 0, wherein if m is 0, then represents a chiral centre;

R³ is (CH₂)ₐE, wherein a is 0, 1 or 2 and E is a group selected from:

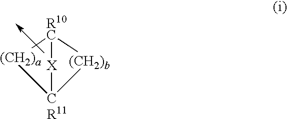

wherein:
X is O, S, NR¹², (CH₂)ᵥ or a bond;
a is 1, 2, 3 or 4;
b is 1, 2 or 3;
v is 1 or 2;
R¹⁰ and R¹¹ are each independently H or C₁₋₄ alkyl; and
R¹² is H, C₁₋₄ alkyl, C(O)C₁₋₄ alkyl, SO₂—C₁₋₆alkyl;
and wherein one or more pairs of hydrogen atoms on adjacent carbon or nitrogen atoms may be replaced by a corresponding number of double bonds, provided the ring system is not aromatic;
(ii) a carbocyclic spiro group containing 6 to 12 carbon atoms;

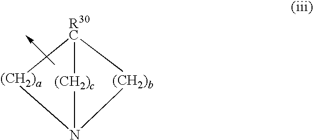

wherein:
a is 1, 2, 3 or 4;
b is 1, 2 or 3;
c is 1 or 2; and
R³⁰ is H or C₁₋₄ alkyl;
and wherein one or more pairs of hydrogen atoms on adjacent carbon atoms may be replaced by a corresponding number of double bonds, provided the ring system is not aromatic;

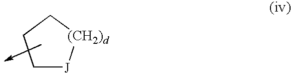

wherein:
d is 0, 1, 2 or 3;
J is SO, SO₂ or NR⁴⁰; and
R⁴⁰ is H, C₁₋₆alkyl, C(O)C₁₋₄ alkyl, SO₂—C₁— alkyl;
and wherein one or more pairs of hydrogen atoms on adjacent carbon atoms may be replaced by a corresponding number of double bonds, provided the ring system is not aromatic;

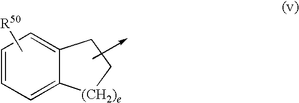

wherein:
e is 0, 1, 2 or 3; and
   R⁵⁰ is H, C₁₋₈alkyl, C₁₋₈alkoxy, OH, halo, CF₃, OCF₃, SCF₃, hydroxy-C₁₋₆alkyl, C₁₋₄alkoxy-C₁₋₆alkyl and C₁₋₄alkyl-S—C₁₋₄-alkyl;
(vi) —CH(cyclopropane)₂;
(vii) C₁₋₆alkyl or C₃₋₈cycloalkyl-C₁₋₆alkyl, wherein the C₁₋₆alkyl moiety is substituted by at least one substituent independently selected from C₁₋₆alkoxy, OH, halo, CF₃, OCF₃, SCF₃, CN, CF₂CF₃, CF₂—C₁₋₄alkyl, hydroxy-C₁₋₆alkyl, C₁₋₄alkoxy-C₁₋₆alkyl and C₁₋₄alkyl-S—C₁₋₄alkyl; and the cycloalkyl group is optionally substituted by at least one substituent independently selected from C₁₋₈alkyl, C₁₋₆alkoxy, OH, halo, CF₃, OCF₃, SCF₃, CN, CF₂CF₃, CF₂—C₁₋₄-alkyl, hydroxy-C₁₋₆alkyl, C₁₋₄alkoxy-C₁₋₆alkyl and C₁₋₄alkyl-S—C₁₋₄alkyl;

aryl and aryl¹ are each independently phenyl, naphthyl, anthracyl or phenanthryl;

heteroaryl is an aromatic 5- or 6-membered heterocycle which contains at least one N, O or S heteroatom, optionally fused to an aryl group; and

het and het¹ are each independently an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom.

In an embodiment of the invention, R¹ is H and R², R³ and m are as defined above.

In a further embodiment of the invention, m is 0 and R¹, R² and R³ are as defined above. Where m is 0, represents the R or S enantiomeric configuration. Thus, in a further embodiment, m is 0, R¹, R² and R³ are as defined above and * represents the S enantiomer.

In a still further embodiment, R¹, R³ and m are as defined above and R² is phenyl, naphthyl or quinolinyl, each optionally substituted by at least one substituent independently selected from C₁₋₈alkyl, C₁₋₈alkoxy, aryl¹, het¹, Oaryl¹, Ohet¹, Saryl¹, Shet¹, OH, halo, CF₃, CHF₂, OCHF₂, OCF₃, SCF₃, CF₂CF₃, CH₂CF₃, CF₂CH₃, hydroxy-C₁₋₆alkyl, C₁₋₆cycloalkyl, C₁₋₆cycloalkyl-C₁₋₄alkyl, OC₁₋₆cycloalkyl, SC₁₋₆cycloalkyl, C₁₋₄alkoxy-C₁₋₆alkyl and C₁₋₄alkyl-S—C₁₋₄alkyl, wherein the aryl¹ and het¹ groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃.

In a further embodiment, R² is substituted by at least one substituent as defined above.

In a yet further embodiment, the substituents may be selected from halo, OH, C₁₋₄alkyl, C₁₋₄alkoxy and CF₃. Optionally, the phenyl, naphthyl or quinolinyl group may be substituted by one, two or three substituents, each independently selected from halo, OH and C₁₋₄alkyl. In a further embodiment, R² is phenyl and is substituted by two substituents selected from chloro, fluoro, OH and C₁₋₄alkyl. In a still further embodiment, R² is dichlorophenyl.

In a yet further embodiment of the invention, there is provided a compound of Formula II

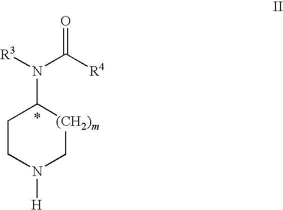

and pharmaceutically and/or veterinarily acceptable derivatives thereof, wherein,

R³ is as defined above;

R⁴ is phenyl, naphthyl, or quinolinyl, each optionally substituted by at least one substituent independently selected from C₁₋₆alkyl, C₁₋₄alkoxy, OH, halo, CF₃, OCF₃, SCF₃, phenoxy, hydroxy —C₁₋₆alkyl, C₁₋₄alkoxy-C₁₋₆alkyl, SC₁₋₆alkyl and C₁₋₄alkyl-S—C₁₋₄alkyl; and

m is 0 or 1, wherein if m is 0, then * represents the R or S enantiomer.

In a further embodiment, R⁴ is phenyl, 1-naphthyl or 2-naphthyl, each optionally substituted by at least one substituent independently selected from C₁₋₄alkyl, C₁₋₄alkoxy, OH, halo, CF₃, OCF₃, SCF₃, phenoxy, hydroxy-C₁₋₆alkyl, C₁₋₄alkoxy-C₁₋₆alkyl, S—C₁₋₄alkyl and C₁₋₄alkyl-S—C₁₋₄alkyl. Preferably R⁴ is phenyl. The substituents may optionally be selected from C₁₋₆alkyl, C₁₋₆alkoxy, halo, OH, phenoxy, S—C₁₋₄alkyl and CF₃. R⁴ may be substituted by one, two or three substituents. Preferably R⁴ is substituted by one or two substituents. In a further embodiment, halo represents chloro or fluoro.

In a yet still further embodiment, m is 0; In such an embodiment, * represents the R or S enantiomer. In a further embodiment, m is 0 and * represents the S enantiomer.

In a still further embodiment, there is provided a compound of Formula III

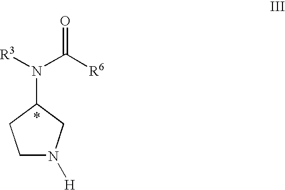

and pharmaceutically and/or veterinarily acceptable derivatives thereof,
wherein:

R³ is as defined above;

R⁶ is phenyl, naphthyl or quinolinyl, each optionally substituted by at least one substituent independently selected from halo, OH, C₁₋₆alkyl, C₁₋₆alkoxy, phenoxy, S—C₁₋₆alkyl and CF₃; and

* represents the R or S enantiomer.

In a further embodiment, R⁶ is phenyl, optionally substituted by at least one substituent independently selected from halo, OH, C₁₋₆alkyl, C₁₋₆alkoxy, phenoxy, S—C₁₋₄alkyl, Oaryl¹ and CF₃. The substituents may optionally be selected from chloro, fluoro, C₁₋₄alkyl, OMe, CF₃, S—C₁₋₄alkyl, Oaryl¹ and OH. The phenyl group may be substituted by one, two or three substituents, in particular one or two substituents.

In a further embodiment, R⁶ is napthyl, optionally substituted by at least one substituent independently selected from halo, OH, C₁₋₆alkyl, C₁₋₆alkoxy, phenoxy, S—C₁₋₄alkyl, Oaryl¹ and CF₃. The substituents may optionally be selected from chloro, fluoro, C₁₋₄alkyl, OMe, CF₃, S—C₁₋₄alkyl, Oaryl¹ and OH. The phenyl group may be substituted by one, two or three substituents, in particular one or two substituents.

In a yet further embodiment, * represents the S enantiomer.

In a further embodiment, the invention provides a compound selected from:
N-bicyclo[2.2.1]hept-2-yl-4-chloro-2-methoxy-N-[(3S)-pyrrolidin-3-yl]benzamide,
N-bicyclo[2.2.1]hept-2-yl-2-chloro-N-[(3S)pyrrolidin-3-yl]benzamide,
N-[(1R,5S)bicyclo[3.2.0]hept-3-yl]-2,3-dichloro-N-[(3S)-pyrrolidin-3-yl]benzamide,
2,3-dichloro-N-(dicyclopropylmethyl)-N-[(3S pyrrolidin-3-yl]benzamide,
2,3-dichloro-N-[(3S)-pyrrolidin-3-yl]-N-(3,3,3-trifluoropropyl)benzamide,
N-bicyclo[2.2.1]hept-2-yl-N-[(3S)-pyrrolidin-3-yl]-2-(trifluoromethyl)benzamide,
2-phenoxy-N-[(3S)-pyrrolidin-3-yl]-N-(3,3,3-trifluoropropyl)benzamide,
2-phenoxy-N-[(3S)-pyrrolidin-3-yl]-N-(2,2,2-trifluoroethyl)benzamide,
N-(2,2-difluoropropyl)-2-phenoxy-N-[(3S)-pyrrolidin-3-yl]benzamide,
N-(2,2-difluoroethyl)-2-phenoxy-N-[(3S)-pyrrolidin-3-yl]benzamide,
N-(2-methoxyethyl)-2-phenoxy-N-[(3S)-pyrrolidin-3-yl]benzamide,
2,4-dichloro-N-[(3S)-pyrrolidin-3-yl]-N-(3,3,3-trifluoropropyl)benzamide,
3,4-dichloro-N-[(3S)pyrrolidin-3-yl]-N-(3,3,3-trifluoropropyl)benzamide,
2,3-dichloro-N-[(3S)-pyrrolidin-3-yl]-N-(2,2,2-trifluoroethyl)benzamide,
2-(ethylthio)-N-[(3S)-pyrrolidin-3-yl]-N-(3,3,3-trifluoropropyl)benzamide,
   and pharmaceutically and/or veterinarily acceptable derivatives thereof.

The above described embodiments of the invention may be combined with one or more further embodiments such that further embodiments are provided wherein two or more variables are defined more specifically in combination. For example, within the scope of the invention is a further embodiment wherein the variables R¹, R², R³, R⁴, R⁶, m and n all have the more limited definitions assigned to them in the more specific embodiments described above. All such combinations of the more specific embodiments described and defined above are within the scope of the invention.

By pharmaceutically and/or veterinarily acceptable derivative it is meant any pharmaceutically or veterinarily acceptable salt, solvate, ester or amide, or salt or solvate of such ester or amide, complex, polymorph, stereoisomer, geometric isomer, tautomeric form, or isotopic variation, of the compounds of formula (I), (II) or (III) or any other compound which upon administration to the recipient is capable of providing (directly or indirectly) a compound of formula (I), (II) or (III) or an active metabolite or residue thereof. Preferably, pharmaceutically acceptable derivatives are salts, solvates, esters and amides of the compounds of formula (I), (II) or (III). More preferably, pharmaceutically acceptable derivatives are salts and solvates.

For pharmaceutical or veterinary use, the salts referred to above will be the pharmaceutically or veterinarily acceptable salts, but other salts may find use, for example in the preparation of compounds of formula (I), (II) or (III) and the pharmaceutically or veterinarily acceptable salts thereof.

The aforementioned pharmaceutically or veterinarily acceptable salts include the acid addition and base salts thereof.

Suitable acid addition salts are formed from acids which form non-toxic salts. Examples include the acetate, aspartate, benzoate, besylate, bicarbonate/carbonate, bisulphate/sulphate, camsylate, citrate, hemicitrate, edisylate, hemiedisylate, esylate, fumarate, gluceptate, gluconate, glucuronate, hibenzate, hydrochloride/chloride, hydrobromide/bromide, hydroiodide/iodide, isethionate, lactate, malate, maleate, malonate, mesylate, methylsulphate, 2-napsylate, nicotinate, nitrate, orotate, pamoate, phosphate/hydrogen phosphate/dihydrogen phosphate, saccharate, stearate, succinate, tartrate and tosylate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of formula (I), (II) or (III) may be readily prepared by mixing together solutions of the compound and the desired acid or base, as appropriate. The salt may precipitate from solution and be collected by filtration or may be recovered by evaporation of the solvent. The degree of ionisation in the salt may vary from completely ionised to almost non-ionised.

Pharmaceutically acceptable solvates in accordance with the invention include hydrates and solvates of the compounds of formula (I), (II) or (III).

Also within the scope of the invention are complexes such as clathrates, drug-host inclusion complexes wherein, in contrast to the aforementioned solvates, the drug and host are present in stoichiometric or non-stoichiometric amounts. Also included in this invention are complexes of the pharmaceutical drug which contain two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of formula (I), (II) or (III) may be modified to provide pharmaceutically or veterinarily acceptable derivatives thereof at any of the functional groups in the compounds. Examples of such derivatives are described in: Drugs of Today, Volume 19, Number 9, 1983, pp 499-538; Topics in Chemistry, Chapter 31, pp 306-316; and in “Design of Prodrugs” by H. Bundgaard, Elsevier, 1985, Chapter 1 (the disclosures in which documents are incorporated herein by reference) and include: esters, carbonate esters, hemi-esters, phosphate esters, nitro esters, sulfate esters, sulfoxides, amides, sulphonamides, carbamates, azo-compounds, phosphamides, glycosides, ethers, acetals and ketals.

It will be further appreciated by those skilled in the art, that certain moieties, known in the art as “pro-moieties”, for example as described by H. Bundgaard in “Design of Prodrugs” (ibid) may be placed on appropriate functionalities when such functionalities are present within compounds of the invention.

The compounds of formula (I), (II) or (III) may contain one or more chiral centres, by virtue of the asymmetric carbon atom defined by certain meanings of R¹ to R⁹ (e.g. s-butyl), or the value of the integer m. Such compounds exist in a number of stereoisomeric forms (e.g. in the form of a pair of optical isomers, or enantiomers). It is to be understood that the present invention encompasses all isomers of the compounds of the invention, including all geometric, tautomeric and optical forms, and mixtures thereof (e.g. tautomeric or racemic mixtures).

The compounds of the invention may exist in one or more tautomeric forms. All tautomers and mixtures thereof are included in the scope of the present invention. For example, a claim to 2-hydroxypyridinyl would also cover its tautomeric form α-pyridonyl.

It is to be understood that the present invention includes radio labelled compounds of formula (I), (II) or (III).

The compounds of formula (I), (II) or (III) and their pharmaceutically and veterinarily acceptable derivatives thereof may also be able to exist in more than one crystal form, a characteristic known as polymorphism. All such polymorphic forms (“polymorphs”) are encompassed within the scope of the invention. Polymorphism generally can occur as a response to changes in temperature or pressure or both, and can also result from variations in the crystallisation process. Polymorphs can be distinguished by various physical characteristics, and typically the x-ray diffraction patterns, solubility behaviour, and melting point of the compound are used to distinguish polymorphs.

Unless otherwise indicated, any alkyl group may be straight or branched and is of 1 to 8 carbon atoms, such as 1 to 6 carbon atoms or 1 to 4 carbon atoms, for example a methyl, ethyl, n-propyl, i-propyl, n-butyl, i-butyl, s-butyl or t-butyl group. Where the alkyl group contains more than one carbon atom, it may be unsaturated. Thus, the term C₁₋₆alkyl includes C₂₋₆ alkenyl and C₂₋₆ alkynyl. Similarly, the term C₁₋₈ alkyl includes C₂₋₄ alkenyl and C₂₋₈ alkynyl, and the term C₁₋₄ alkyl includes C₂₋₄ alkenyl and C₂₋₄ alkynyl.

The term halogen is used to represent fluorine, chlorine, bromine or iodine.

Unless otherwise indicated, the term het includes any aromatic, saturated or unsaturated 4-, 5 or 6-membered heterocycle which contains up to 4 heteroatoms selected from N, O and S. Examples of such heterocyclic groups included furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, tetrahydropyranyl, pyridyl, piperidinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazapinyl, thiazepinyl, diazepinyl and thiazolinyl. In addition, the term heterocycle includes fused heterocyclyl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, dihydroquinazdinyl, benzothiazolyl, phthalimido, benzodiazepinyl, indolyl and isoindolyl. The terms het, heterocyclyl and heterocyclic should be similarly construed.

For the avoidance of doubt, unless otherwise indicated, the term substituted means substituted by one or more defined groups. In the case where groups may be selected from a number of alternative groups, the selected groups may be the same or different. Further, the term independently means that where more than one substituent is selected from a number of possible substituents, those substituents may be the same or different.

Hereinafter, the compounds of formula (I), (II) or (III) and their pharmaceutically and veterinarily acceptable derivatives, the radio labelled analogues of the foregoing, the isomers of the foregoing, and the polymorphs of the foregoing, are referred to as “compounds of the invention”.

In one embodiment of the invention, the compounds of the invention are the pharmaceutically and veterinarily acceptable derivatives of compounds of formula (I), (II) or (III), such as the pharmaceutically or veterinarily acceptable salts or solvates of compounds of formula (I), (II) or (III), (e.g. pharmaceutically or veterinarily acceptable salts of compounds of formula (I), (II) or (III)).

In a still further embodiment of the invention, there is provided a compound of the invention which is an inhibitor of serotonin and/or noradrenaline monoamine re-uptake, having SRI or NRI Ki values of 500 nM or less, preferably 200 nM or less. In a further embodiment, the compound has SRI and/or NRI Ki values of 100 nM or less. In a yet further embodiment, the compound has SRI and/or NRI Ki values of 50 nM or less. In a still yet further embodiment, the compound has SRI and/or NRI Ki values of 25 nM or less.

Without wishing to be bound by theory, it is believed that for certain of the diseases or conditions for which the compounds of the invention are indicated it is useful for the compound to be a more potent inhibitor of the reuptake of one of serotonin or noradrenaline than the other. Thus, in an embodiment of the invention, the reuptake of noradrenaline is inhibited to greater degree than the reuptake of serotonin. In an alternative embodiment, the reuptake of serotonin is inhibited to a greater degree than the reuptake of noradrenaline. For example, in the treatment of pain, it is believed that compounds of the invention which inhibit the reuptake of noradrenaline have good efficacy. Thus, an embodiment of the invention provides a method of treating pain which comprises administering to a patient in need of such treatment a therapeutically effective amount of a compound according to the invention which is capable of inhibiting the reuptake of noradrenaline. In this embodiment, the compound of the invention may selectively inhibit the reuptake of noradrenaline or it may inhibit the reuptake of noradrenaline preferentially to the inhibition of serotonin reuptake or it may inhibit the reuptake of serotonin preferentially to the inhibition of noradrenaline reuptake. In a further embodiment of the invention, there provided compounds which are more potent noradrenalin reuptake inhibitors than serotonin reuptake inhibitors. Accordingly, such an embodiment of the invention provides a method of treating pain which comprises administering to a patient in need of such treatment a therapeutically effective amount of a compound according to the invention which is capable of inhibiting the reuptake of noradrenaline to a greater extent than the reuptake of serotonin.

According to Scheme 1, compounds of Formula (V) may be prepared from compounds of Formula (VI) by reaction with an aldehyde R^{3′}CHO or a suitable ketone, followed by reaction with an acid or acid chloride R²COX (where X is OH or halo), or an acid anhydride, optionally further followed by deprotection.

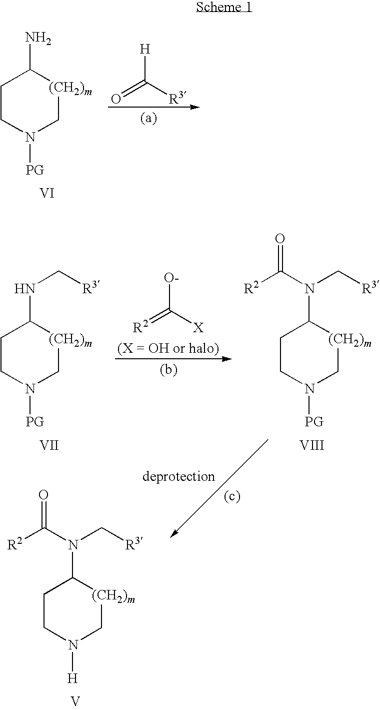

In the above scheme, R² and m are as defined above, PG is a protecting group and the moiety —CH₂R^{3′} satisfies the definition of R³ when a is 1 and R^{3′} is E.

(a)—Reductive Amination

The reaction of the 1° amine (VI) with the aldehyde to form the 2° amine (VII) is a reductive amination reaction, in which the dehydration of the amine and the aldehyde is followed by reduction of the formed imine by a metal hydride reagent or hydrogenation, in a suitable solvent at room temperature.

In this reaction, equimolar amounts of amine and aldehyde are typically treated with either sodium triacetoxyborohydride (STAB), NaCN(BH)₃ or NaBH₄, in a suitable solvent (e.g. DCM, THF) at room temperature for 1 to 24 hours. Alternatively, an excess of a reducing agent (e.g. NaBH₄, LiAlH₄, STAB) in a suitable solvent (e.g. THF, MeOH, EtOH) is added after the amine and aldehyde have been mixed for 1-18 hours, optionally in the presence of a drying agent (e.g. molecular sieve) or with the removal of water using Dean-Stark apparatus with a suitable solvent (e.g. toluene, xylene). A further alternative involves catalytic hydrogenation in the presence of a palladium or nickel catalyst (e.g. Pd/C, Raney® Ni) under an atmosphere of H₂, optionally at elevated temperature and pressure, in a suitable solvent (e.g. EtOH).

A more specific example of the reductive amination involves treatment of the aldehyde with the amine in the presence of either 10% Pd/C, optionally in the presence of triethylamine, in ethanol under about 415 kPa (about 60 psi) of hydrogen at room temperature for 18 hours, or an excess of sodium borohydride in methanol at room temperature for 6 hours.

(b)—Amide Formation

The formation of a peptide linkage between the acid, acid halide or other suitable carbonyl-containing group and the amine (VII) may be undertaken by using either:

(i) the acid halide (or acid or anhydride) and the amine (VII), with an excess of acid acceptor in a suitable solvent, or
(ii) the acid, optionally with a conventional coupling agent, and the amine (VII), optionally in the presence of a catalyst, with an excess of acid acceptor in a suitable solvent.

Examples of such reactions are as follows:
(i) An acid chloride (optionally generated in-situ) is reacted with an excess of the amine (VII), optionally with an excess of 30 amine such as Et₃N, Hünig's base or NMM, in DCM or dioxane, optionally at elevated temperature for 1 to 24 hrs;
(ii) An acid, WSCDI/DCCI/TBTU and HOBT/HOAT is reacted with an excess of amine (VII) and an excess of NMM, Et₃N, Hünig's base in THF, DCM or EtOAc, at rt. for 4 to 48 hrs; or
(iii) An acid and PYBOP®/PyBrOP®/Mukaiyama's reagent is reacted with an excess of amine (VII) and an excess of NMM, Et₃N, Hünig's base in THF, DCM or EtOAc, at rt. for 4 to 24 hrs.

Where the acid halide is an acid chloride (i.e. X═Cl), this may be generated in-situ by standard methodology and then reacted with the amine (VII) in the presence of triethylamine in dichloromethane at 70° C. for 90 minutes.

(c)—Deprotection

Where PG is a suitable amine-protecting group, preferably BOC, trifluoroacetate, benzyloxycarbonyl or benzyl, the removal of PG from (VII), to form the unprotected amine (V), is performed by a method selective to the protecting group as detailed in “Protective Groups in Organic Synthesis”, 3^{rd} edition, by TW Greene and PGM Wuts. John Wiley and Sons, Inc., 1999, incorporated herein by reference.

Examples of such deprotection reactions are as follows:

When PG is BOC, the deprotection involves treatment of (VIII) with an excess of strong acid (e.g. HCl, TFA) at room temperature in a suitable solvent (e.g. DCM, EtOAc, dioxan).

When PG is trifluoroacetate, the deprotection involves treatment of (VIII) with a base (e.g. K₂CO₃, Na₂CO₃, NH₃, Ba(OH)₂) in an alcoholic solvent (e.g. MeOH, EtOH), optionally with water and optionally at elevated temperature.

When PG is Bn or Bz, the deprotection involves either transfer hydrogenation with a transition metal or transition metal salt hydrogenation catalyst (e.g. Pd/C, Pd(OH)₂) in the presence of a hydrogen donor (e.g. NH₄⁺HCO₂⁻) in a polar solvent (e.g. tetrahydrofuran, ethanol, methanol) optionally at elevated temperature and/or pressure, or catalytic hydrogenation in the presence of a palladium or nickel catalyst (e.g. Pd/C, Raney® Ni) under an atmosphere of H₂, optionally at elevated temperature and pressure, in a suitable solvent.

More specifically:

When PG is BOC, the deprotection involves treatment with either an excess of 4M hydrochloric acid in dioxan for 18 hours at room temperature. Or with TFA in DCM for 4.5 hours at RT.

When PG is trifluoroacetate, the deprotection involves treatment with K₂CO₃ in methanol:water mixture (5:1 to 10:1) at room temperature for 18 hours.

When PG is Bn or Bz, the deprotection involves treatment with NH₄⁺HCO₂⁻ and 10% Pd/C in ethanol under gentle reflux for between 6 and 20 hours.

Alternative methods for preparing the secondary amine compound (VII) from the primary amine compound (VI) are described in Schemes 1a and 1b below.

In Schemes 1a and 1b, the value of m is shown as 0 and the value of n is 1. However, it will be immediately apparent to those skilled in the art that corresponding reagents, where m is 1 or where n is 2, can be used in the following schemes to prepare the appropriate end compounds.

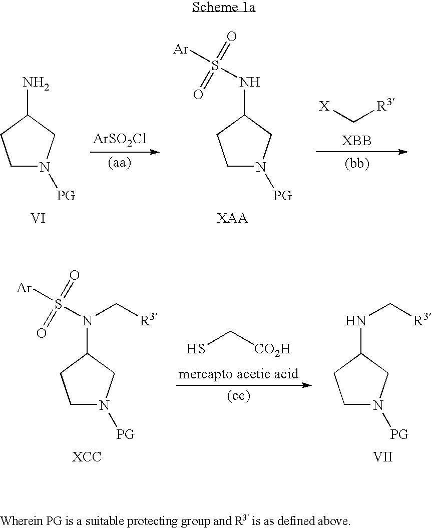

According to scheme 1a, compounds of formula VII can be prepared from compounds of formula VI by reaction with a sulfonyl chloride, followed by alkylation of the resulting sulfonyl amide, and then removal of the sulfonyl moiety.

(aa) Preparation of the sulfonyl amide. Reaction of equimolar amounts of the primary amine (VI) and a sulfonyl chloride such as 2,4-dinitrobenzenesulphonyl chloride in a suitable solvent (such as DCM, THF or Toluene) in the presence of an organic base (such as pyridine or 2,6-lutidine) or an inorganic base (such as a carbonate salt) for up to 24 hours affords the sulfonylamide (XAA).

(bb) Alkylation of Sulfonylamide XAA. The sulfonylamide of formula XAA is alkylated using an activated alkylating agent XBB, where X is a leaving group such as a halogen (e.g. iodo, bromo or chloro) or a sulfonyl ester (such as methanesulfonate) in the presence of an organic or an inorganic base, in a suitable solvent (such as DMF or THF). Alternatively the alkylation of sulfonylamide of formula XAA can be achieved using an alcohol XBB (where X is OH), a phosphine (such as triphenyl phosphine) and an azodicarboxylate compound (such as DIAD) in a suitable solvent, such as THF, for up to 24 hours at a temperature between −20 C and 45 C.

(cc) Removal of the sulfonyl group. A compound of formula XCC is treated with an organic base (such as triethylamine) or an inorganic base (such as a carbonate or a hydroxide) in a suitable solvent (such as DCM, THF or a lower alcohol) and with a thiol (such as mercaptoacetic acid) for up to 24 hours, optionally at an elevated temperature.

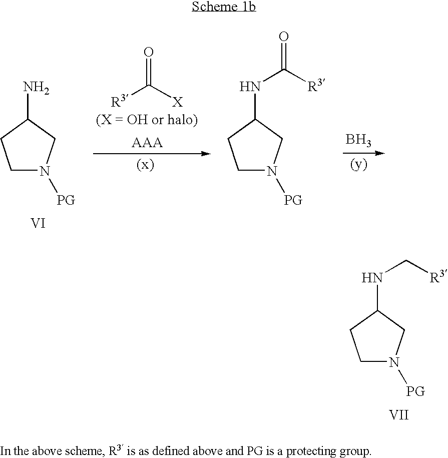

### Acylation-Reduction

According to Scheme 1b, compounds of Formula (VII) may be prepared from 1° amine of Formula (VI) by reaction with a carboxylic acid or acid halide AAA (optionally prepared in-situ) R^{3′}COX (where X is OH or halo), followed by reaction with a reducing agent, such as borane.

(x)—Amide Formation

The formation of an amide bond between the acid or acid halide and the 1° amine (VI) may be undertaken by using either:

(i) the acyl halide (or the acid or acid anhydride) and the amine (VI), with an excess of acid acceptor in a suitable solvent, or

(ii) the acid, optionally with a conventional coupling agent, and the amine (VI), optionally in the presence of a catalyst, with an excess of acid acceptor in a suitable solvent.

Examples of such reactions are as follows:
(iv) An acid chloride (optionally generated in-situ) is reacted with an excess of the amine (VI), optionally with an excess of 30 amine such as Et₃N, Hünig's base or NMM, in DCM or dioxane, optionally at elevated temperature for 1 to 24 hrs;
(v) An acid, WSCDI/DCCI/TBTU and HOBT/HOAT is reacted with an excess of amine (VI) and an excess of NMM, Et₃N, Hünig's base in THF, DCM or EtOAc, at rt. for 4 to 48 hrs; or
(vi) An acid and 1-propyl phosphonic ester cyclic anhydride/PYBOP®/PyBrOP®/Mukaiyama's reagent is reacted with an excess of amine (VI) and an excess of NMM, Et₃N, Hünig's base in THF, DCM or EtOAc, at rt. for 1 to 24 hrs.

A more specific example of the amide formation involves treatment of the acid with the amine in the presence of 1-propyl phosphonic ester cyclic anhydride and in the presence of triethylamine in DCM at room temperature for 1 hour.

Where the acid halide is an acid chloride (i.e. X═Cl), this may be generated in-situ by standard methodology and then reacted with the amine (VI) and triethylamine in dichloromethane at 70° C. for 90 minutes

(y)—Reduction

The reaction (y) is a reduction of the amide to amine (VII), for example by a hydride reducing agent under suitable conditions.

Conveniently, the reduction of the amide can be carried out in the presence of Borane in THF, at reflux, for 2 hours. This is followed by the addition of methanol (optionally with the addition of aqueous ammonium chloride) and further refluxing for between 4 and 48 hours, after which time the amine (VII) is isolated.

According to Scheme 2, compounds of Formula (IX) may be prepared from compounds of Formula (VI) by reaction with R³-L, where L is a leaving group, under suitable conditions. The resulting compound of Formula (IX) may then be converted to a compound of Formula (II) by amide formation and deprotection in a manner analogous to that described above in relation to Scheme 1.

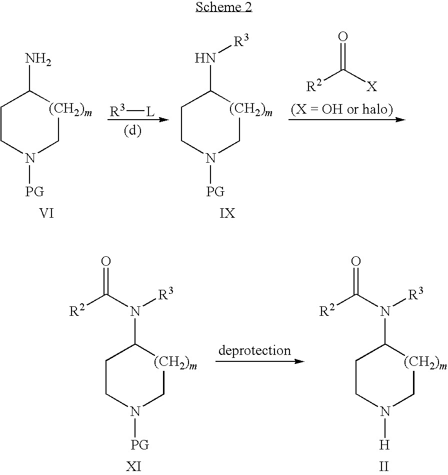

In the above scheme, R², R³ and m are as defined above, PG is a suitable protecting group and L is a leaving group, whose meaning will depend, inter alia, on the nature of the reaction and the specific reaction conditions employed. Suitable leaving groups will be readily apparent to the skilled person and are described in many standard organic chemistry texts, for example: “Advanced Organic Chemistry”, Jerry March, Third Edition, Wiley (1985), page 587, incorporated herein by reference; they include halogen (e.g. Br) and sulfonate esters (e.g. methanesulfonate or trifluoromethanesulfonate).

According to Scheme 3, compounds of Formula (IX) may be prepared from a ketone of Formula (XII) by reaction with a primary amine R³—NH₂ under suitable conditions. The resulting compound of Formula (IX) may then be converted to a compound of Formula (II) by amide formation and deprotection in a manner analogous to that described above in relation to Scheme 1.

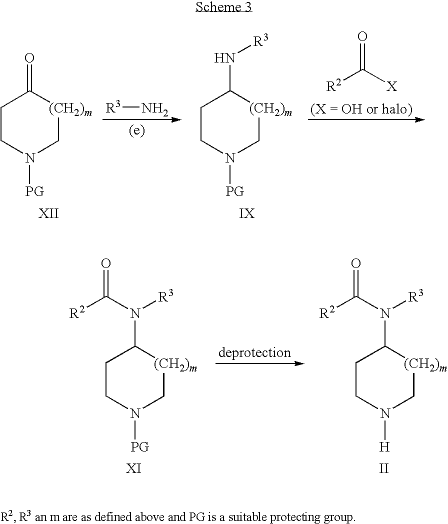

The reaction (e) of the primary amine R³—NH₂ with the ketone (XII) may conveniently be a reductive amination reaction in which the dehydration of the amine and the ketone is followed by reduction of the resultant imine, for example by a metal hydride reagent or hydrogenation, under suitable conditions.

Conveniently, the reaction of the amine and the ketone is carried out in the presence of titanium (IV) tetraisopropoxide in THF at room temperature for 18 hours, followed by reduction by an excess of sodium borohydride in methanol at room temperature for 5 hours.

The skilled person is able to select the most appropriate synthetic route to the desired compound according to Formula (I), (II) or (III). The above schemes may of course be modified as appropriate in accordance with the common general knowledge of those skilled in the art.

For example, the skilled person will of course appreciate that the hydrogen attached to the piperidine or pyrrolidine nitrogen (depending upon the value of m) of the deprotected amide (II) or (V) can be replaced with alternative groups as desired to form a compound of Formula (I) where n is 1 and m is 0 or 1 by the use of conventional synthetic methodologies.

In addition, compounds of Formula (I) where n is 2 and m is 0 can be prepared by analogous processes to those described above using the appropriate starting materials.

It will be appreciated by those skilled in the art that one or more sensitive functional groups may need to be protected and deprotected during the synthesis of a compound of Formula (I), (II) or (III). This may be achieved by conventional techniques, for example as described in “Protective Groups in Organic Synthesis”, 3^{rd} edition, by TW Greene and PGM Wuts. John Wiley and Sons, Inc., 1999, incorporated herein by reference, which also describes methods for the removal of such groups.

It will be apparent to those skilled in the art that certain protected derivatives of compounds of the invention, which may be made prior to a final deprotection stage, may not possess pharmacological activity as such, but may, in certain instances, be administered orally or parenterally and thereafter metabolized in the body to form compounds of the invention which are pharmacologically active. Such derivatives may therefore be described as prodrugs. Further, certain compounds of the invention may act as prodrugs of other compounds of the invention.

Thus, according to a further aspect of the invention, there is provided a process for preparing compounds of Formula (I), (II) or (III), which comprises reacting a compound of formula (X):

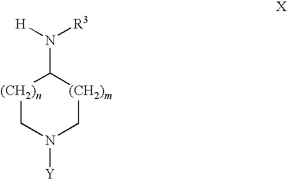

wherein R³, n and m are as defined above and Y is R¹ or a protecting group, with an acid or acyl halide: R²COX, wherein X is OH or halo, and deprotecting if necessary.

Where R³ includes a methylene moiety which is directly bonded to the nitrogen atom, then the compound of Formula (X) may be prepared by reacting a compound of Formula (XXI) with an aldehyde R^{3′}CHO (wherein —CH₂R^{3′} satisfies the definition of R³ when a is 1 and R^{3′} is E).

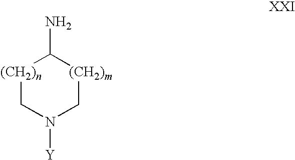

Alternatively, the compound of Formula (X) may be prepared by reacting a compound of Formula (XXI) with a compound R³-L, where L is a leaving group, optionally selected from halide, methanesulfonate and trifluoromethanesulfonate.

Furthermore, the compound of Formula (X) may be prepared by reacting a compound of Formula (XXII) with a compound R³—NH₂

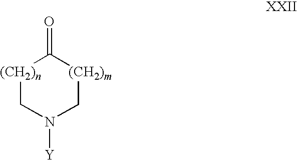

Certain intermediates described above are novel compounds and it is to be understood that all novel intermediates herein form further aspects of the present invention.

Racemic compounds may be separated either using preparative HPLC and a column with a chiral stationary phase, or resolved to yield individual enantiomers utilizing methods known to those skilled in the art. In addition, chiral intermediate compounds may be resolved and used to prepare chiral compounds of the invention.

According to a further aspect of the invention, there is provided one or more metabolites of the compounds of the invention when formed in vivo.

The compounds of the invention may have the advantage that they are more potent, have a longer duration of action, have a broader range of activity, are more stable, have fewer side effects or are more selective, or have other more useful properties than the compounds of the prior art.

The compounds of the invention are useful because they have pharmacological activity in mammals, including humans. Thus, they are useful in the treatment or prevention of disorders in which the regulation of monoamine transporter function is implicated, more particularly disorders in which inhibition of re-uptake of serotonin or noradrenaline is implicated. Furthermore, the compounds of the invention are of use in disorders in which inhibition of both serotonin and noradrenaline is implicated, such as urinary incontinence. Additionally, the compounds of the invention are of use in disorders in which it may be desired to inhibit preferentially the reuptake of one of noradrenaline or serotonin compared with the other, such as pain.

Accordingly the compounds of the invention are useful in the treatment of urinary incontinence, such as genuine stress incontinence (GSI), stress urinary incontinence (SUI) or urinary incontinence in the elderly; overactive bladder (OAB), including idiopathic detrusor instability, detrusor overactivity secondary to neurological diseases (e.g. Parkinson's disease, multiple sclerosis, spinal cord injury and stroke) and detrusor overactivity secondary to bladder outflow obstruction (e.g. benign prostatic hyperplasia (BPH), urethral stricture or stenosis); nocturnal eneuresis; urinary incontinence due to a combination of the above conditions (e.g. stress incontinence associated with overactive bladder); and lower urinary tract symptoms, such as frequency and urgency. The term OAB is intended to encompass both OAB wet and OAB dry.

In view of their aforementioned pharmacological activity the compounds of the invention are also useful in the treatment of depression, such as major depression, recurrent depression, single episode depression, subsyndromal symptomatic depression, depression in cancer patients, depression in Parkinson's patients, postmyocardial infarction depression, paediatric depression, child abuse induced depression, depression in infertile women, post partum depression, premenstrual dysphoria and grumpy old man syndrome.

In view of their aforementioned pharmacological activity the compounds of the invention are also useful in the treatment of cognitive disorders such as dementia, particularly degenerative dementia (including senile dementia, Alzheimer's disease, Pick's disease, Huntingdon's chorea, Parkinson's disease and Creutzfeldt-Jakob disease) and vascular dementia (including multi-infarct dementia), as well as dementia associated with intracranial space occupying lesions, trauma, infections and related conditions (including HIV infection), metabolism, toxins, anoxia and vitamin deficiency; mild cognitive impairment associated with ageing, particularly age associated memory impairment (AAMI), amnestic disorder and age-related cognitive decline (ARCD); psychotic disorders, such as schizophrenia and mania; anxiety disorders, such as generalised anxiety disorder, phobias (e.g. agoraphobia, social phobia and simple phobias), panic disorder, obsessive compulsive disorder, post traumatic stress disorder, mixed anxiety and depression; personality disorders such as avoidant personality disorder and attention deficit hyperactivity disorder (ADHD); sexual dysfunction, such as premature ejaculation, male erectile dysfunction (MED) and female sexual dysfunction (FSD) (e.g. female sexual arousal disorder (FSAD)); premenstrual syndrome; seasonal affective disorder (SAD); eating disorders, such as anorexia nervosa and bulimia nervosa; obesity; appetite suppression; chemical dependencies resulting from addiction to drugs or substances of abuse, such as addictions to nicotine, alcohol, cocaine, heroin, phenobarbital and benzodiazepines; withdrawal syndromes, such as those that may arise from the aforementioned chemical dependencies; cephalic pain, such as migraine, duster headache, chronic paroxysmal hemicrania, headache associated with vascular disorders, headache associated with chemical dependencies or withdrawal syndromes resulting from chemical dependencies, and tension headache; pain; Parkinson's diseases, such as dementia in Parkinson's disease, neuroleptic-induced Parkinsonism and tardive dyskinesias); endocrine disorders, such as hyperprolactinaemia; vasospasm, such as in the cerebral vasculature; cerebellar ataxia; Tourette's syndrome; trichotillomania; kleptomania; emotional lability; pathological crying; sleep disorder (cataplexy); and shock.

In view of their aforementioned pharmacological activity the compounds of the invention are also useful in the treatment of a number of other conditions or disorders, including hypotension; gastrointestinal tract disorders (involving changes in motility and secretion) such as irritable bowel syndrome (IBS), ileus (e.g. post-operative ileus and ileus during sepsis), gastroparesis (e.g. diabetic gastroparesis), peptic ulcer, gastroesophageal reflux disease (GORD, or its synonym GERD), flatulence and other functional bowel disorders, such as dyspepsia (e.g. non-ulcerative dyspepsia (NUD)) and non-cardiac chest pain (NCCP); hot flashes; and fibromyalgia syndrome.

The compounds of the invention, being serotonin and/or noradrenaline reuptake inhibitors are potentially useful in the treatment of a further range of disorders, including pain.

Physiological pain is an important protective mechanism designed to warn of danger from potentially injurious stimuli from the external environment. The system operates through a specific set of primary sensory neurones and is activated by noxious stimuli via peripheral transducing mechanisms (see Millan, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually in twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a heightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, 2000, Science, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid 40′ arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhoea, cystitis and pancreatitis and pelvic pain.

It should be noted that some types of pain have multiple aetiologies and thus can be classified in more than one area, e.g. back pain and cancer pain have both nociceptive and neuropathic components.

Other types of pain include:
pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache, tension-type headache mixed headache and headache associated with vascular disorders; and
orofacial pain, including dental pain, otic pain, burning mouth syndrome and temporomandibular myofascial pain.

Disorders of particular interest include urinary incontinence, such as mixed incontinence, GSI and USI; pain; depression; anxiety disorders, such as obsessive-compulsive disorder and post traumatic stress disorder; personality disorders, such as ADHD; sexual dysfunction; and chemical dependencies and withdrawal syndromes resulting from chemical dependencies.

Thus, according to further aspects, the invention provides:
i) a compound of the invention for use in human or veterinary medicine;
ii) a compound of the invention for use in the treatment of a disorder in which the regulation of monoamine transporter function is implicated, such as urinary incontinence;
iii) the use of a compound of the invention in the manufacture of a medicament for the treatment of a disorder in which the regulation of monoamine transporter function is implicated;
iv) a compound of the invention for use in the treatment of a disorder in which the regulation of serotonin or noradrenaline is implicated;
v) the use of a compound of the invention in the manufacture of a medicament for the treatment of a disorder in which the regulation of serotonin or noradrenaline is implicated;
vi) a compound of the invention for use in the treatment of a disorder in which the regulation of serotonin and noradrenaline is implicated;
vii) the use of a compound of the invention in the manufacture of a medicament for the treatment of a disorder in which the regulation of serotonin and noradrenaline is implicated;
viii) a compound of the invention for use in the treatment of pain or urinary incontinence, such as GSI or USI;
ix) the use of a compound of the invention in the manufacture of a medicament for the treatment of pain or urinary incontinence, such as GSI or USI;
x) a method of treatment of a disorder in which the regulation of monoamine transporter function is implicated which comprises administering a therapeutically effective amount of a compound of the invention to a patent in need of such treatment;
xi) a method of treatment of a disorder in which the inhibition of the reuptake of serotonin or noradrenaline is implicated which comprises administering a therapeutically effective amount of a compound of the invention to a patient in need of such treatment;
xii) a method of treatment of a disorder in which the inhibition of the reuptake of serotonin and noradrenaline is implicated which comprises administering a therapeutically effective amount of a compound of the invention to a patient in need of such treatment; and
xiii) a method of treating pain or urinary incontinence, such as GSI or USI, which comprises administering a therapeutically effective amount of a compound of the invention to a patient in need of such treatment.

It is to be appreciated that all references herein to treatment include curative, palliative and prophylactic treatment, unless explicitly stated otherwise.

The compounds of the invention may be administered alone or as part of a combination therapy. If a combination of therapeutic agents is administered, then the active ingredients may be administered either sequentially or simultaneously in separate or combined pharmaceutical formulations.

Examples of suitable agents for adjunctive therapy include:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcydizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone; an alpha-adrenergic, e.g. doxazosin, tamsulosin, clonidine, guanfacine, dexmetatomidine, modafinil, phentolamine, terazasin, prazasin or 4-amino-6,7-dimethoxy-2-(5-methane-sulfonamido-1,2,3,4-tetrahydroisoquinol-2-yl)-5-(2-pyridyl)quinazoline;
a tricylic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5 bis(trifluoromethyl)benzyl]-8,9,10,11-tetrahydro-9-methyl-5-(4-methylphenyl-7H-[1,4]diazocino[2,1-g][1,7]-naphthyridine-6-13-dione (TAK-437), 5-[[(2R,3S)-2-[(1R) 1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, terniverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)N-methyl-4-(3-pyridinyl)-3-buten-1-amine (RJR-2403), (R)-5-(2-azetidinylmethoxy)-2-chloropyridine (ABT-594) or nicotine; Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-4-ethyl-piperazin-1-yl-1-sulphonyl)phenyl]-5-methyl-7-propyl-3H-imidazo[5,1-f][1,2,4]triazin-4-one (vardenafil), 5-(5-acetyl-2-butoxy-3-pyridinyl)-3-ethyl-2-1-ethyl-3-azetidinyl-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R-3-amino-5-methyl-heptanoic acid, (3S,5R-3-amino-5-methyl-octanoic acid, (2S,4S)-4-(3-chlorophenoxy)proline, (2S,4S)-4-(3-fluorobenzyl)proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitaiopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalane), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethyldomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)₃ pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulflde;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-6504);
a sodium channel blocker, such as lidocaine;
a 5-HT3 antagonist, such as ondansetron, granisetron, tropisetron, azasetron, dolasetron or alosetron;
an oestrogen agonist or selective oestrogen receptor modulator (e.g. HRT therapies or lasofoxifene);
an alpha-adrenergic receptor agonist, such as phenylpropanolamine or R-450;
a dopamine receptor agonist (e.g. apomorphine, teachings on the use of which as a pharmaceutical may be found in U.S. Pat. No. 5,945,117), including a dopamine D2 receptor agonist (e.g. premiprixal, Pharmacia Upjohn compound number PNU95666; or ropinirole);
a PGE1 agonist (e.g. alprostadil);
   and the pharmaceutically acceptable salts and solvates thereof.

The invention thus provides, in a further aspect, a combination comprising a compound of the invention together with a further therapeutic agent.

For human use the compounds of the invention can be administered alone, but in human therapy will generally be administered in admixture with a suitable pharmaceutical excipient, diluent or carrier selected with regard to the intended route of administration and standard pharmaceutical practice.

For example, the compounds of the invention, can be administered orally, buccally or sublingually in the form of tablets, capsules (including soft gel capsules), ovules, elixirs, solutions or suspensions, which may contain flavouring or colouring agents, for immediate-, delayed-, modified-, sustained-, dual-, controlled-release or pulsatile delivery applications. The compounds of the invention may also be administered via intracavemosal injection. The compounds of the invention may also be administered via fast dispersing or fast dissolving dosage forms.

Such tablets may contain excipients such as microcrystalline cellulose, lactose, sodium citrate, calcium carbonate, dibasic calcium phosphate, glycine, and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention, and their pharmaceutically acceptable salts, may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intravenously, intra-arterially, intraperitoneally, intrathecally, intraventricularly, intraurethrally, intrasternally, intracranially, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

For oral and parenteral administration to human patients, the daily dosage level of the compounds of the invention or salts or solvates thereof will usually be from 10 to 500 mg (in single or divided doses).

Thus, for example, tablets or capsules of the compounds of the invention or salts or solvates thereof may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including PE), compounds of the invention may be taken as a single dose on an “as required” basis (i.e. as needed or desired).

### Example Tablet Formulation

In general a tablet formulation could typically contain between about 0.01 mg and 500 mg of a compound according to the present invention (or a salt thereof) whilst tablet fill weights may range from 50 mg to 1000 mg. An example formulation for a 10 mg tablet is illustrated:

| Ingredient | % w/w |
|---|---|
| Free base or salt of compound | 10.000* |
| Lactose | 64.125 |
| Starch | 21.375 |
| Croscarmellose Sodium | 3.000 |
| Magnesium Stearate | 1.500 |

*This quantity is typically adjusted in accordance with drug activity and is based on the weight of the free base.

The compounds of the invention can also be administered intranasally or by inhalation and are conveniently delivered in the form of a dry powder inhaler or an aerosol spray presentation from a pressurised container, pump, spray or nebulizer with the use of a suitable propellant, e.g. dichlorodifluoromethane, trichlorofluoromethane, dichlorotetra-fluoro-ethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compounds of the invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compounds of the invention can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch. They may also be administered by the ocular, pulmonary or rectal routes.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or, more of the following: mineral oil, liquid petrolatum, white petrolatum, propylene glycol, polyoxyethylene-polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters, wax, cetearyl alcohol, 2-octyldodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

For oral or parenteral administration to human patients the daily dosage levels of compounds of formula (I), and their pharmaceutically acceptable salts, will be from 0.01 to 30 mg/kg (in single or divided doses) and preferably will be in the range 0.01 to 5 mg/kg. Thus tablets will contain 1 mg to 0.4 g of compound for administration singly or two or more at a time, as appropriate. The physician will in any event determine the actual dosage which will be most suitable for any particular patient and it will vary with the age, weight and response of the particular patient. The above dosages are, of course only exemplary of the average case and there may be instances where higher or lower doses are merited, and such are within the scope of the invention.

Oral administration is preferred.

For veterinary use, a compound of the invention is administered as a suitably acceptable formulation in accordance with normal veterinary practice and the veterinary surgeon will determine the dosing regimen and route of administration which will be most appropriate for a particular animal.

Thus according to a further aspect, the invention provides a pharmaceutical formulation containing a compound of the invention and a pharmaceutically acceptable adjuvant, diluent or carrier.

The combinations referred to above may also conveniently be presented for use in the form of a pharmaceutical formulation and thus pharmaceutical formulations comprising a combination as defined above together with a pharmaceutically acceptable adjuvant, diluent or carrier comprise a further aspect of the invention. The individual components of such combinations may be administered either sequentially or simultaneously in separate or combined pharmaceutical formulations.

When a compound of the invention is used in combination with a second therapeutic the dose of each compound may differ from that when the compound is used alone. Appropriate doses will be readily appreciated by those skilled in the art.

The invention is illustrated by the following non-limiting examples in which the following abbreviations and definitions may be used:

APCI Atmospheric pressure chemical ionisation
Arbocel® filter agent

### br Broad

BOC tert-butoxycarbonyl
CDI carbonyldiimidazole
δ chemical shift
d doublet
Δ heat
DCCI dicyclohexylcarbodiimide
DCM dichloromethane

### DMF N,N-dimethylformamide

DMSO dimethylsulfoxide
ES⁺ electrospray ionisation positive scan
ES⁻ electrospray ionisation negative scan
h hours
HOAT 1-hydroxy-7-azabenzotriazole
HOBT 1-hydroxybenzotriazole
HPLC high pressure liquid chromatography
m/z mass spectrum peak
min minutes
MS mass spectrum
NMM N-methyl morpholine
NMR nuclear magnetic resonance
q quartet
s singlet
t triplet
m multiplet
TBTU 2-(1H-benzotriazol-1-yl)-1,1,3,3-tetramethyluronium tetrafluoroborate
Tf trifluoromethanesulfonyl
TFA trifluoroacetic acid
THF tetrahydrofuran
TLC thin layer chromatography
TS⁺ thermospray ionisation positive scan
WSCDI 1-(3-dimethylaminopropyl)-3-ethylcarbodiimide hydrochloride

The Preparations and Examples that follow illustrate the invention but do not limit the invention in any way. All starting materials are available commercially or described in the literature. All temperatures are in ° C. Flash column chromatography was carried out using Merck silica gel 60 (9385). Solid Phase Extraction (SPE) chromatography was carried out using Varian Mega Bond Elut (Si) cartridges (Anachem) under 15 mmHg vacuum. Thin layer chromatography (TLC) was carried out on Merck silica gel 60 plates (5729). Melting points were determined using a Gallenkamp MPD350 apparatus and are uncorrected. NMR was carried out using a Varian-Unity Inova 400 MHz NMR spectrometer or a Varian Mercury 400 MHz NMR spectrometer. Mass spectroscopy was carried out using a Finnigan Navigator single quadrupole electrospray mass spectrometer or a Finnigan aQa APCI mass spectrometer.

Where it is stated that compounds were prepared in the manner described for an earlier Preparation or Example, the skilled person will appreciate that reaction times, number of equivalents of reagents and reaction temperatures may be modified for each specific reaction, and that it may nevertheless be necessary or desirable to employ different work-up or purification conditions.

### PREPARATION 1

### tert-Butyl (3S)-3-[(1R,3r,5S)bicyclo[3.2.0]hept-3-ylamino]pyrrolidine-1-carboxylate

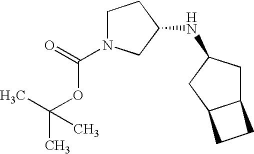

tert-butyl (3S)-3-aminopyrrolidine-1-carboxylate (2.3 g, 12.4 mmol) was added to a solution of bicyclo[3.2.0]heptan-3-one (1.5 g, 13.6 mmol, as prepared according to WO 2001028978) and 10% Pd/C (500 mg), in ethanol (50 ml), and the reaction mixture was left at 50° C. under about 345 kPa (about 50 psi) of hydrogen gas, for 16 hours. The reaction mixture was filtered through Arbocel®, and washed through thoroughly with ethyl acetate. The filtrate was concentrated in vacuo and the crude product was purified by column chromatography on silica gel using a gradient of Methylene Chloride changing to Methylene Chloride:Methanol:ammonia (100:10:1 by volume) to yield the title product, 1.29, (35%) as a cis-cyclopentyl compound (determined by nOe correlations).

¹HNMR (CD3OD, 400 MHz) δ: 1.38-1.42 (m, 2H), 1.45 (s, 9H), 1.72-1.78 (m, 3H), 2.12 (m, 1H), 2.20-2.27 (m, 4H), 2.62 (m, 2H), 3.00-3.06 (m, 2H), 3.28 (m, 1H), 3.38 (m, 1H), 3.46 (m, 1H), 3.57 (m, 1H); MS APCI⁺ m/z 281 [MH]⁺

### PREPARATION 2

### tert-Butyl (3S)-3-[(1R,3r,5S)bicyclo[3.2.0]hept-3-yl(2,3-dichlorobenzoyl)amino]pyrrolidine-1-carboxylate

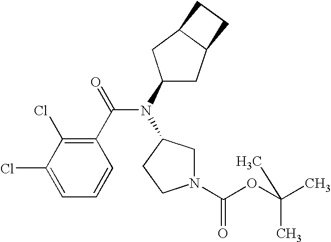

The amine of preparation 1 (500 mg, 1.78 mmol) was added to a solution of N-methyl morpholine (360 mg, 3.56 mmol), in toluene (30 ml), and the reaction mixture was treated with 2,3-dichloro-benzoyl chloride (450 mg, 2.13 mmol). The reaction mixture was then heated to 70° C. and stirred for 3 hours, after which time it was washed with a 10% citric acid solution. The organic phase was separated, dried over magnesium sulfate and concentrated in vacuo. The crude product was purified by column chromatography on silica gel using a gradient of methylene chloride, changing to methylene chloride:methanol (95:5 by volume) affording the title product as the cis compound, 660 mg, (81%).

¹HNMR (CD3OD, 400 MHz, rotamers) δ: 1.42 (s, 2.5H), 1.48 (s, 6.5H), 1.75-1.80 (m, 3H), 1.92-1.96 (m, 2H), 2.09 (m, 1H), 2.22 (m, 3H), 2.45-2.51 (m, 1.5H), 2.75 (m, 2H), 3.47 (m, 1H), 3.64-3.72 (m, 2.5H), 3.89 (m, 1H), 4.07 (m, 0.5H), 4.31 (m, 0.5H), 7.2 (m, 1H), 7.38 (m, 1H), 7.59 (m, 1H); MS APCI+ m/z 453 [MH]* and 353 [(M-Boc)H]⁺

### PREPARATION 3

### tert-Butyl (3S)-3-(bicyclo[2.2.1]hept-2-ylamino)pyrrolidine-1-carboxylate

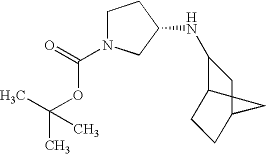

tert-Butyl (3S)-3-(bicyclo[2.2.1]hept-2-ylamino)pyrrolidine-1-carboxylate was prepared by a method similar to that described for preparation 1 using tertbutyl (3S)-3-aminopyrrolidine-1-carboxylate and norcamphor to yield the desired product, 7.32 g-(97%, solid) as a mixture of diastereoisomers.

¹HNMR (CDCl₃, 400 MHz) δ: 0.61 (m, 1H), 1.07 (m, 1H), 1.19 (m, 1H), 1.33 (m, 3H), 1.45 (s, 9H), 1.51 (m, 1H), 1.60-1.65 (m, 2H), 1.92 (m, 1H), 2.02 (m, 1H), 2.14 (brs, 1H), 2.24 (brd, 1H), 2.99-3.07 (m, 2H), 3.22-3.31 (m, 2H), 3.41-3.60 (m, 2H); MS APCI⁺ m/z 281 [MH]⁺.

### PREPARATION 4

### 4-Chloro-2-methoxybenzoyl chloride

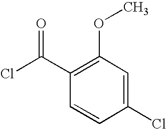

4-Chloro-2-methoxy-benzoic acid (14.9 g, 79.8 mmol) was suspended in dichloromethane (180 ml), cooled in an ice bath, and then the cooled solution was treated with oxalyl chloride (13.9 ml, 159.6 mmol) and N,N-dimethylformamide (10 drops). The ice was removed and the reaction mixture was warmed to room temperature. It was then stirred at room temperature for a further 3 hours. The reaction mixture was then concentrated in vacuo and azeotroped with dichloromethane to provide the title compound, 16.6 g (100%) as a white solid.

¹HNMR (CDCl₃, 400 MHz) δ: 3.93 (s, 3H), 7.00 (s, 1H), 7.04 (d, 1H), 8.04 (d, 1H); MS APCI⁺ m/z 201 [M-methyl ester H]⁺.

### PREPARATION 5

### tert-Butyl (3S)-3-[bicycio[2.2.1]hept-2-yl(4-chloro-2-methoxybenzoyl)amino]pyrrolidine-1-carboxylate

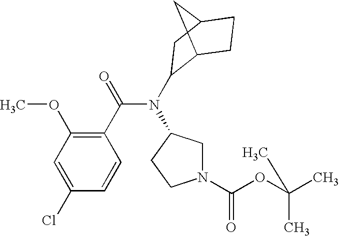

The benzoyl chloride from preparation 4 (1.25 g, 6.11 mmol) was added to a solution of N-methylmorpholine (1.03 g, 10.19 mmol) and the amine of preparation 3 (1.5 g, 5.35 mmol), in toluene (50 ml). The reaction mixture was heated at 50° C. overnight. It was then washed with a 10% aqueous solution of citric acid, twice with a 1N sodium hydroxide solution and then with brine. The organic layer was dried over magnesium sulfate and concentrated in vacuo. The crude product was purified by column chromatography on silica gel using methylene chloride:methanol:ammonia (100:10:1 by volume) affording the title product as an oil (1.01 g, 42%) as a mixture of diastereoisomers.

¹HNMR (CD3OD, 400 MHz, rotamers) δ: 1.18 (m, 2H), 1.46 (brs, 12H), 1.65-1.73 (m, 3H), 2.04-2.25 (m, 4H), 2.44 (m, 0.5H), 2.72 (m, 0.5H), 3.38 (m, 1H), 3.62 (m, 2H), 3.79 (m, 1H), 3.84 (s, 3H), 4.05 (m, 1H), 7.03 (m, 1H), 7.10 (m, 1H), 7.16 (m, 1H); MS APCI⁺ m/z 449 [MH]⁺.

### PREPARATION 6

### tert-Butyl (3S)-3-{bicyclo[2.2.1]hept-2-yl[2-(trifluoromethyl)benzoyl]amino)pyrrolidine-1-carboxylate

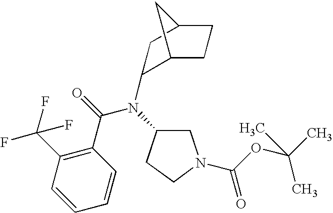

tert-Butyl (3S)-3-bicyclo[2.2.1]hept-2-yl[2-(trifluoromethyl)benzoyl]amino}pyrrolidine-1-carboxylate was prepared by a method similar to that described for preparation 5 using the amine of preparation 3, 2-(trifluoromethyl)benzoyl chloride and 4-dimethylaminopyridine (10 mol %), to yield the desired product, 540 mg (37%) as a mixture of diastereoisomers.

¹HNMR (CDCl₃, 400 MHz) δ: 1.04 (m, 0.5H), 1.09 (m, 1H), 1.18 (m, 1H), 1.36-1.40 (m, 2.5H), 1.46 (s, 9H), 1.64-1.75 (m, 2.5H), 1.97 (m, 0.5H), 2.05 (m, 1H), 2.23 (m, 1H), 2.67 (m, 0.5H), 2.93 (m, 1H), 3.36 (m, 1H), 3.45 (m, 0.5H), 3.58-3.83 (m, 4.5H), 4.14 (m, 0.5H), 7.29 (m, 1H), 7.47 (m, 1H), 7.56 (m, 1H), 7.67 (m, 1H); MS APCI⁺ m/z 453 [MH]⁺.

### PREPARATION 7

### tert-Butyl (3S)-3-{bicyclo[2.2.1]hept-2-yl[2-chlorobenzoyl]amino}pyrrolidine-1-carboxylate

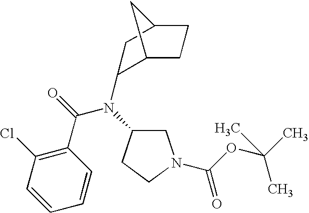

tert-Butyl (3S)-3-{bicyclo[2.2.1]hept-2-yl[2-chlorobenzoyl]amino}pyrrolidine-1 carboxylate was prepared by a method similar to that described for preparation 5 using the amine of preparation 3, 2-, chlorobenzoyl chloride and dimethylaminopyridine (10 mol %) to yield the desired product, 940 mg (72%) as a mixture of diastereoisomers.

¹HNMR (CDCl₃, 400 MHz, rotamers) δ: 1.09-1.16 (m, 2H), 1.24-1.29 (m, 3H), 1.26 (s, 9H), 1.63-1.68 (m, 2H), 1.87 (m, 0.5H), 2.00 (m, 1H), 2.12 (m, 0.5H), 2.26 (m, 1.5H), 2.70 (m, 0.5H), 2.92 (m, 0.5H), 3.39 (m, 2H), 3.61-3.74 (m, 3H), 3.88 (m, 1H), 4.14 (m, 0.5H), 7.23 (m, 1H), 7.28-7.30 (m, 2H), 7.37 (m, 1H); MS APCI⁺ m/z 419 [MH]⁺.

### PREPARATION 8

### tert-Butyl (3S-3-[(3,3,3-trifluoropropanoyl)amino]pyrrolidine-1-carboxylate

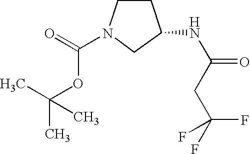

3,3,3-Trifluoropropionic acid (1.76 g, 13.74 mmol) was added to a solution of tert-butyl (3S)-3-aminopyrrolidine-1-carboxylate (2.33 g, 12.50 mmol), in dichloromethane (63 ml), at room temperature. Triethylamine (4.36 ml, 31.25 mmol) was then added. The reaction was then cooled to 0° C. and 2,4,6-tripropyl-1,3,5,2,4′,6-trioxa triphosphinane 2,4,6-trioxide (50% w/w in ethyl acetate, 8.9 ml, 13.75 mmol) was added. The mixture was then stirred at room temperature for 1.5 hours and then concentrated under reduced pressure. The residue was diluted in dichloromethane (200 ml) and a 20% aqueous potassium carbonate solution (40 ml) added. The mixture was stirred at room temperature for 18 hours and the layers separated. The organic layer was washed with more potassium carbonate solution (500 ml), brine, dried over magnesium sulfate and concentrated in vacuo to provide the title compound as a yellow solid (3.70 g, 94%).

¹HNMR (CDCl₃, 400 MHz) δ: 1.43 (s, 9H), 1.82-1.90 (m, 1H), 2.11 (m, 1H), 3.05 (q, 2H), 3.16-3.25 (m, 1H), 3.35-3.42 (m, 2H), 3.56 (m, 1H), 4.46 (m, 1H), 6.09 (brs, 0.5H), 6.36 (brs, 0.5H).

MS APCI+m/z 297 [MH]⁺.

### PREPARATION 9

### tert-Butyl (3S)-3-[(3,3,3-trifluoropropyl)amino]pyrrolidine-1-carboxylate

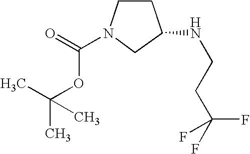

Borane (1M in tetrahydrofuran, 10 ml, 10.14 mmol) was added slowly to a solution of the compound of Preparation 8 (1 g, 3.38 mmol), in tetrahydrofuran (10 ml). The mixture was heated at reflux for 2 hours. Methanol was then added carefully to quench the reaction mixture and the mixture was concentrated in vacuo. The residue was taken up in dichloromethane and minimal volume of methanol for dissolution and purified by column chromatography on silica gel using a gradient of ethyl acetate changing to ethyl acetate:pentane (30:70 by volume) affording the borane-complex of the title product. This was then taken up in methanol (20 ml) and heated at reflux for 1 hour. The reaction mixture was concentrated in vacuo and azeotroped with toluene to yield the title product as a clear oil (800 mg, 84%).

¹HNMR (CD3OD, 400 MHz) δ: 1.45 (s, 9H), 1.75 (m, 1H), 2.08 (m, 1H), 2.32-2.41 (m, 2H), 2.79-2.85 (m, 2H), 3.08 (m, 1H), 3.30-3.33 (m, 2H), 3.44 (m, 1H), 3.53 (m, 1H); MS APCI+m/z 283 [MH]⁺.

### PREPARATION 10

### tert-Butyl (3S)-3-[(2,3-dichlorobenzoyl)(3,3,3-trifluoropropyl)amino]pyrrolidine-1-carboxylate

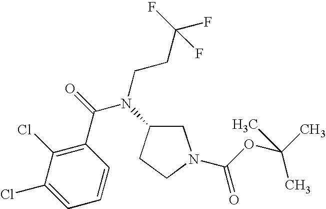

tert-Butyl (3S-3-[(2,3-dichlorobenzoyl)(3,3,3-trifluoropropyl)amino]pyrrolidine-1-carboxylate was prepared by a method similar to that described for Preparation 2 using the amine of Preparation 9 and 2,3-dichlorobenzoyl chloride to yield the desired product (482 mg, 95%).

¹HNMR (CDCl₃, 400 MHz, rotamers) δ: 1.44 (d, 9H), 1.90 (m, 1H), 2.15 (m, 1H), 2.45 (m, 1H), 2.81 (m, 1H), 3.10-3.18 (m, 1.5H), 3.28 (m, 0.5H), 3.35-3.44 (m, 1.5H), 3.52-3.59 (m, 1.5H), 3.71 (m, 0.5H), 3.84 (m, 0.5H), 4.03 (m, 1H), 7.14 (m, 1H), 7.30 (m, 1H), 7.53 (m, 1H); MS APCI+ m/z 455 [MH]⁺.

### PREPARATION 11

### tert-Butyl (3S-3-[(2-phenoxybenzoyl)(3,3,3-trifluoropropyl)amino]pyrrolidine-1-carboxylate

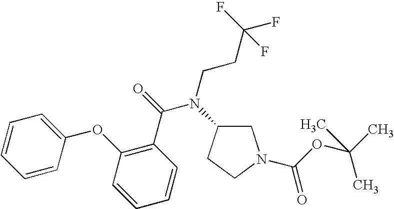

The title compound was prepared by a method similar to that described for Preparation 2 using the amine of Preparation 9 and 2-phenoxybenzoyl chloride (prepared from the acid by a method similar to that described for Preparation 4) to yield the desired product (294 mg, 69%).

¹HNMR (CDCl₃, 400 MHz, rotamers) δ: 1.44 (2×s, 9H), 1.89-1.95 (m, 2H), 2.19 (m, 1H), 2.48-2.65 (m, 1H), 3.10-3.17 (m, 2H), 3.30 (m, 1H), 3.52 (m, 1H), 3.65 (m, 1H), 3.80 (m, 1H), 4.32 (m, 1H), 6.92-6.97 (m, 3H), 7.14 (m, 1H), 7.14 (m, 1H), 7.32-7.39 (m, 4H); MS APCI+ m/z 479 [MH]⁺.

### PREPARATION 12

### tert-Butyl (3S)-3-[(2,2-difluoropropanoyl)amino]pyrrolidine-1-carboxylate

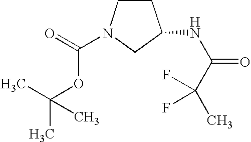

Triethylamine (950 μl, 6.8 mmol) was added to a solution of 2,2-difluoropropanoic acid (300 mg, 2.7 mmol) and tert-butyl (3S-3-aminopyrrolidine-1-carboxylate (461 mg, 2.5 mmol) in dichloromethane (15 ml), at room temperature. 2,4,6-Tripropyl-1,3,5,2,4,6-trioxa triphosphinane 2,4,6-trioxide (50% w/w in ethyl acetate, 1.75 ml, 2.7 mmol) was then added and the reaction mixture was stirred at room temperature for 17 hours. The reaction mixture was diluted with 10% potassium carbonate solution (100 ml) and then extracted with dichloromethane (100 ml). The organic phase was dried over magnesium sulfate and concentrated in vacuo to provide the title compound as a clear oil (609 mg, 95%).

¹HNMR (CDCl₃, 400 MHz) δ: 1.42 (s, 9H), 1.74-1.98 (m, 4H), 2.17-2.23 (m, 1H), 3.18-3.52 (m, 3H), 3.61-3.71 (m, 1H), 4.43 (m, 1H), 6.39 (br s, 1H).

### PREPARATION 13

### tert-Butyl (3S)-3-[(2,2-difluoropropyl)amino]pyrrolidine-1-carboxylate

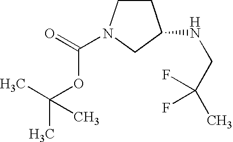

The title compound was obtained as a clear oil in quantitative yield from the compound of preparation 12 following a similar procedure to that described for preparation 9.

¹HNMR (CD3OD, 400 MHz) δ: 1.43 (s, 9H), 1.57-1.63 (m, 3H), 1.70-1.79 (m, 1H), 2.01-2.11 (m, 1H), 2.87-2.98 (m, 2H), 3.10 (m, 1H), 3.30 (m, 1H), 3.36 (m, 1H), 3.40-3.58 (m, 2H); MS APCI+ m/z 265 [MH]⁺.

### PREPARATION 14

### tert-butyl (3S)-3-[(2,2-difluoropropyl)(2-phenoxybenzoyl)amino]pyrrolidine-1-carboxylate

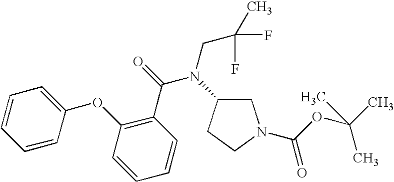

A solution of the amine from preparation 13 (583 mg, 2.2 mmol) and triethylamine (641 μl, 3.5 mmol), in dichloromethane (10 ml), was added to 2-phenoxybenzoyl chloride (803 mg, 3.5 mmol) and the solution was stirred at room temperature for 18 hours. The solution was then concentrated under reduced pressure and the residue was re-dissolved in ether (40 ml). The solution was washed with 10% citric acid solution (50 ml), 2M sodium hydroxide solution (50 ml), then dried over magnesium sulfate and evaporated under reduced pressure. The residue was purified by column chromatography on silica gel using a gradient of methylene chloride, changing to methylene chloride:methanol:ammonia (98:1.8:0.2 by volume) to afford the title compound, 770 mg.

¹HNMR (CD3OD, 400 MHz, rotamers) δ: 1.40-1.63 (m, 12H), 1.90-2.21 (m, 2H), 3.05-4.42 (m, 7H), 6.91-7.02 (m, 3H), 7.12-7.24 (m, 2H), 7.32-7.50 (m, 4H).

### PREPARATION 15

### tert-butyl (3S)-3-[(trifluoroacetyl)amino]pyrrolidine-1-carboxylate

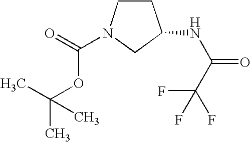

2.2 g (11.8 mmol, 1 eq.) of tert-butyl (3S)-3-aminopyrrolidine-1-carboxylate and 4 ml (excess) of ethyl trifluoroacetate were dissolved in 40 ml of ethanol. The solution was stirred at room temperature overnight, after which time 2 ml of ethyl trifluoroacetate were added and the solution was heated at 60° C. for 3 hours. The solvent was removed under reduced pressure, affording 3.67 g (crude) of the title compound as a yellow oil. The crude compound was used directly in the next step.

### PREPARATION 16

### tert-butyl (3S-3-[(2,2,2-trifluoroethyl)amino]pyrrolidine-1-carboxylate

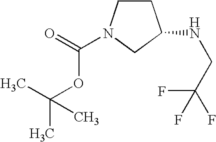

The crude compound from preparation 15 was dissolved in 20 ml of dry THF, and 48 ml of 1M borane-THF solution, in THF, was added dropwise. The solution was stirred at reflux overnight, then cooled and 50 ml of aqueous ammonium chloride were added drop wise. The mixture was then stirred at 60° C. for 2 hours. The THF was evaporated under reduced pressure, the aqueous phase was extracted three times with 100 ml of ethyl acetate. The combined organic phases were washed with brine, dried over magnesium sulfate and concentrated under reduced pressure, affording 3.04 g of a clear oil. This oil was purified by column chromatography on silica gel using a gradient of methylene chloride, changing to methylene chloride:methanol:ammonia (100:10:1 by volume), affording 2.44 g of the title product as an oil (77%).

¹HNMR (CD3OD, 400 MHz) δ: 1.43 (s, 9H), 1.70-1.80 (m, 1H), 2.00-2.10 (m, 1H), 3.05-3.55 (m, 7H); MS APCI+ m/z 269 [MH]⁺.

### PREPARATION 17

### tert-butyl (3S)-3-[(difluoroacetyl)amino]pyrrolidine-1-carboxylate

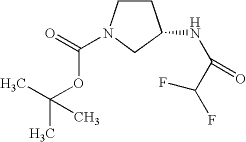

Tert-butyl (3S)-3-aminopyrrolidine-1-carboxylate (2.24 g, 12 mmol), difluoroacetic acid (0.836 ml, 13.2 mmol) and triethylamine (4.2 ml, 30 mmol) were combined in dichloromethane (80 ml). A solution of 2,4,6-Tripropyl-1,3,5,2,4,6-trioxa triphosphinane 2,4,6-trioxide (50% w/w in ethyl acetate, 8.6 ml, 13.2 mmol) was added and the reaction mixture was then stirred at room temperature for 20 hours. The solution was diluted with dichloromethane (150 ml), washed with 2M sodium hydroxide, dried over magnesium sulfate, filtered and evaporated in vacuo. The title compound was obtained as a yellow oil (3.6 g, 13.6 mmol, 100%).

¹H-NMR (CDCl₃, 400 MHz): δ 1.46 (s, 9H), 2.22 (m, 1H), 3.11 (m, 1H), 3.27 (b, 1H), 3.45 (b, 2H), 3.64 (m, 1H), 4.48 (m, 1H), 5.87 (t, 1H), 6.39 (b, 1H); MS APCI⁺ m/z 264 [MH]⁺

### PREPARATION 18

### tert-butyl (3S)-3-[(2,2-difluoroethyl)amino]pyrrolidine-1-carboxylate

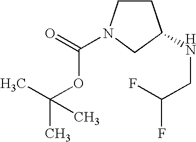

To a solution of compound of preparation 17 (3.6 g, 13.6 mmol), in tetrahydrofuran (130 ml), stirring at 0° C., was added portionwise a 1M solution of borane in tetrahydrofuran (40 ml, 40 mmol). The reaction mixture was heated at 65° C. for 3 hours, then stirred at room temperature, for a further 18 hours. The mixture was quenched with an aqueous solution of ammonium chloride and stirred at room temperature for a further 44 hours. The solution was then diluted with ethyl acetate (100 ml), washed with a 10% aqueous solution of potassium carbonate (2×200 ml), dried over magnesium sulfate, filtered and evaporated in vacuo. The residue was dissolved in methanol and heated at 75° C. for 2.5 hours. The solvent was evaporated in vacuo to yield the title compound as a colourless oil (2.7 g, 10.8 mmol, 83%).

¹H-NMR (CD₃OD, 400 MHz): δ 1.42 (s, 9H), 1.73 (m, 1H), 2.03 (m, 1H), 3.92 (m, 2H), 3.06 (m, 1H), 3.27-3.38 (m, 2H), 3.29-3.57 (m, 2H), 5.83 (tt, 1H); MS APCI⁺ m/z 251 [MH]⁺

### PREPARATION 19

### tert-butyl (3S)-3-[(2-phenoxybenzoyl)(2,2,2-trifluoroethyl)amino]pyrrolidine-1-carboxylate

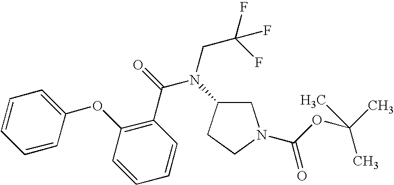

The title compound was prepared from 0.87 g (3.24 mmol, 1.0 eq.) of the compound of preparation 16, and 2-phenoxybenzoyl chloride (0.90 g, 3.89 mmol, 1.2 eq.) following a similar procedure to that described for preparation 5 (using triethylamine instead of N-Methyl Morpholine). Purification by column chromatography on silica gel using a gradient of methylene chloride changing to methylene chloride:methanol:ammonia (100:10:1 by volume) afforded 1.01 g the title product as an oil (67%).

¹HNMR (CD₃OD, 400 MHz) δ: 1.40 (s, 9H), 1.80-2.20 (br m, 2H), 3.0-3.2 (br, 2H), 3.30-3.40 (br m, 2H), 4.05-4.40 (br m, 3H), 6.80-7.60 (m, 9H); MS APCI⁺ m/z 465 [MH]⁺.

### PREPARATION 20

### tert-butyl (3S)-3-[(2,2-difluoroethyl)(2-phenoxybenzoyl)amino]pyrrolidine-1-carboxylate

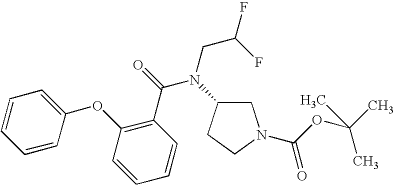

The title compound was prepared from 0.50 g (2.00 mmol, 1.0 eq.) of the compound of preparation 18, and 2-phenoxybenzoyl chloride (0.56 g, 2.40 mmol, 1.2 eq.) following a similar procedure to that described for preparation 5 (using triethylamine instead of N-methyl morpholine). 0.73 g (82%) of the title compound was obtained, as an oil.

¹HNMR (CD₃OD, 400 MHz) δ: 1.40 (s, 9H), 1.80-2.20 (br m, 2H), 3.0-3.90 (br m, 7H), 4.40 (br m, 1H), 6.80-7.60 (m, 9H); MS APCI⁺ m/z 447 [MH]⁺.

### PREPARATION 21

### Tert-butyl (3S)-3-[(dicyclopropylmethyl)amino]pyrrolidine-1-carboxylate

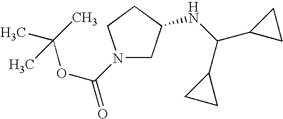

To a solution of tert-butyl (3S)-3-aminopyrrolidine-1-carboxylate (1 g, 5.36 mmol) in ethanol (40 ml) was added dicyclopropyl ketone (0.66 ml, 5.9 mmol) and titanium isopropoxide (7.86 ml, 26.8 mmol), and the reaction mixture was stirred at room temperature for 72 hours. Sodium borohydride (406 mg, 10.72 mmol) was added and the reaction mixture was stirred at room temperature for a further 18 hours. The mixture was then quenched with water (2 ml) and the ethanol was evaporated in vacuo. The residue was partitioned between 1M sodium hydroxide (30 ml) and dichloromethane (50 ml), and the remaining solid was removed by filtration. The aqueous phase was extracted with dichloromethane (2×50 ml) and the combined organic phases were dried over magnesium sulphate, filtered and evaporated in vacuo. The crude material was purified by column chromatography using an ISCO® silica cartridge eluting with dichloromethane to dichloromethane:methanol:0.880 ammonia (90:10:1). The title compound was obtained as a colourless oil (77 mg, 0.27 mmol, 5%).

¹H-NMR (CDCl₃, 400 MHz,): δ 0.13 (m, 2H), 0.23 (m, 2H), 0.49 (d, 4H), 0.84 (m, 2H), 1.22 (t, 1H), 1.45 (s, 9H), 1.65 (m, 1H), 2.07 (m, 1H), 3.00 (m, 1H), 3.30 (m, 1H), 3.45 (m, 1H), 3.63 (m, 2H); MS APCI⁺ m/z 281 [MH]⁺

### PREPARATION 22

### Tert-butyl (3S)-3-[(2,3-dichlorobenzoyl)(dicyclopropylmethyl)amino]pyrrolidine-1-carboxylate

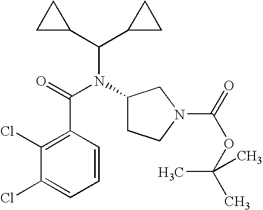

To a solution of the compound of preparation 21 (65 mg, 0.232 mol) in dichloromethane (1 ml) was added triethylamine (0.065 ml, 0.464 mmol) and 2,3-dichlorobenzoyl chloride (63 mg, 0.302 mmol), and the reaction mixture was then stirred at room temperature for 72 hours. The solvent was evaporated in vacuo and the residue was redissolved in toluene (1 ml). Triethylamine (0.065 ml, 0.464 mmol) was added and the reaction mixture was heated at 60° C. for 8 hours. The mixture was filtered through a hydrophobic filter and the solvent was evaporated in vacuo. The crude material was purified by column chromatography using an ISCO® silica cartridge eluting with 0-35% ethyl acetate: pentane (50 mg, 0.11 mmol, 47%).

¹H-NMR (CDCl₃, 400 MHz): δ 0.10-0.39 (m, 4H), 0.46 (m, 2H), 0.60 (m, 2H), 0.69-0.90 (m, 1H), 0.98 (m, 1H), 1.47 (s, 9H), 2.09 (m, 1H), 2.50 (m, 1H), 2.88-3.02 (m, 1H), 3.41 (quartet, 1H), 3.61-3.76 (m, 2H), 3.99 (quintet, 1H), 4.17 (m, 1H), 7.08 (t, 1H), 7.21 (m, 1H), 7.44 (m, 1H); MS APCI⁺ m/z 397 [M-^{t}Bu]⁺

### PREPARATION 23

### tert-butyl (3S-3-[(methoxyacetyl)amino]pyrrolidine-1-carboxylate

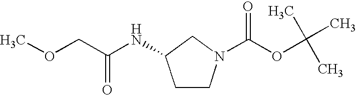

Methoxyacetyl chloride (4.2 ml, 46.1 mmol) was added dropwise to a solution of tert-butyl (3S)-3-aminopyrrolidine-1-carboxylate (7.15 g, 38.4 mmol) and triethylamine (10.7 ml, 76.8 mmol) in dichloromethane (150 ml), at room temperature, under nitrogen. After stirring for 18 hours, the solvent was removed by evaporation under reduced pressure. The residue was dissolved in diethyl ether and washed with 5% w/w aqueous citric acid. The aqueous phase was extracted with dichloromethane and the combined organic phases were dried over magnesium sulphate. The mixture was then filtered and solvent was removed by evaporation under reduced pressure to produce a brown oil (10.0 g).

¹HNMR (CDCl₃, 400 MHz) δ: 1.46 (s, 9H), 1.85 (m, 1H), 2.16 (m, 1H), 3.20 (m, 1H), 3.44 (m, 5H), 3.63 (m, 1H), 3.88 (s, 2H), 4.49 (m, 1H), 6.56 (m, 1H); MS APCI⁺ m/z 259 [MH]⁺

### PREPARATION 24

### tert-butyl (3S-3-[(2-methoxyethyl)amino]pyrrolidine-1-carboxylate

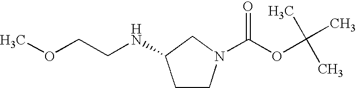

Borane (1M in tetrahydrofuran, 115 ml, 115 mmol) was added to a solution of the compound from preparation 23 (10.0 g, 38.4 mmol) in anhydrous tetrahydrofuran (115 ml), under nitrogen. After stirring the reaction mixture at reflux for 4 hours, it was then cooled to room temperature and quenched with methanol. The solvent was removed by evaporation under reduced pressure and the residue was re-dissolved in methanol (150 ml) and saturated aqueous ammonium chloride. After heating the solution at reflux for 18 hours, under nitrogen, the volume was halved by evaporation under reduced pressure and then diluted with diethyl ether (200 ml). The aqueous phase was then extracted with diethyl ether (200 ml). The combined organic phases were washed with brine (100 ml) and dried over magnesium sulfate. The mixture was filtered and the solvent was removed by evaporation under reduced pressure to produce a colourless oil (6.6 g).

¹HNMR (CDCl₃, 400 MHz) δ: 1.38 (m, 1H), 1.43 (s, 9H), 1.54 (m, 1H), 2.05 (m, 1H), 2.22 (m, 1H), 2.98 (m, 2H), 3.36 (m, 4H), 3.49-3.73 (m, 5H); MS APCI⁺ m/z 245 [MH]⁺

### PREPARATION 25

### tert-butyl (3S)-3-[(2-methoxyethyl)(2-phenoxybenzoyl)amino]pyrrolidine-1-carboxylate

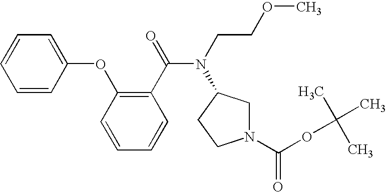

The title compound was prepared by a similar method to that of preparation 20 using the amine of preparation 24 and 2-phenoxybenzoyl chloride (Tetrahedron (1988), 44(18), 5857-60) to produce the desired product, 474 mg (72%) as a colourless oil.

¹HNMR (CDCl₃, 400 MHz) δ: 1.44 (s, 9H), 1.83-2.17 (m, 2H), 3.06-3.74 (m, 1H), 4.29 (m, 1H), 6.91 (m, 1H), 6.97 (m, 2H), 7.15 (m, 2H), 7.31 (m, 4H): MS APCI⁺ m/z 441 [MH]⁺

### PREPARATION 26

### 2-(ethylthio)benzoic acid

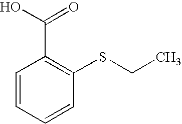

A 1M solution of sodium hydroxide (10 ml) was added to 2-mercaptobenzoic acid (1 g, 6.4 mmol) in ethanol (10 ml), followed by iodoethane (1 g, 6.4 mmol). The reaction mixture was stirred for 72 hours and then the ethanol was evaporated under reduced pressure. The reaction mixture was cooled in an ice bath and acidified to pH 1 with 2N aqueous hydrochloric acid. The precipitate was collected by filtration, washed with water and dried under reduced pressure to afford the title compound, 1.09 g, 98%.

¹HNMR (400 MHz, CD₃OD) δ: 1.32 (t, 3H), 2.9 (q, 2H), 7.13 (t, 1H), 7.38 (d, 1H), 7.43 (t, 1H), 7.88 (d, 1H); MS APCI⁻ m/z 181 [M-H]⁻

### EXAMPLE 1

### N-(1R,3r,5 S)-Bicyclo[3.2.0]hept-3-yl-2,3-dichloro-N-[(3S)-pyrrolidin-3-yl]benzamide

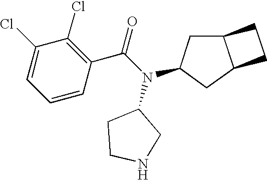

The Boc protected compound of preparation 2 (600 mg, 1.32 mmol) was added to a solution of 4M hydrochloric acid in dioxane (20 ml), and the reaction mixture was stirred at room temperature for 6 hours. The reaction mixture was concentrated in vacuo, then taken up in ethyl acetate and washed with 1N sodium hydroxide solution. The layers were separated and the aqueous layer was extracted again with ethyl acetate. The organic layers were combined, dried over sodium sulfate, filtered and concentrated in vacuo. The crude product was then purified by column chromatography on silica gel using a gradient of methylene chloride, changing to methylene chloride:methanol:ammonia (100:10:1 by volume) to afford the title product (cis compound), 440 mg, (94%).

¹HNMR (CD3OD, 400 MHz) δ: 1.72-1.81 (m, 4H), 1.95 (m, 1H), 2.10 (m, 1H), 2.21-2.26 (m, 2H), 2.46-2.53 (brm, 4H), 3.21 (m, 1H), 3.52 (m, 1H), 3.59-3.76 (m, 3H), 4.52 (m, 1H), 7.28-7.32 (dd, 1H), 7.40 (t, 1H), 7.61 (d, 1H); MS APCI⁺ m/z 353 [MH]⁺

### EXAMPLE 2

### N-Bicyclo[2.2.1]hept-2-yl-4-chloro-2-methoxy-N-[(3S)-pyrrolidin-3-yl]benzamide

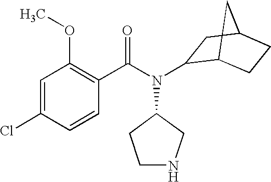

The Boc protected compound of preparation 5 (980 mg, 2.18 mmol) was dissolved in dichloromethane (15 ml), under nitrogen, and the reaction mixture was treated with trifluoroacetic acid (15 ml), which was added drop wise at 0° C. The reaction mixture was then stirred at 0° C. for 3 hours, after which time it was evaporated, azeotroped twice with toluene, and then concentrated in vacuo. The resulting mixture was taken up in ethyl acetate and washed with 1N sodium hydroxide solution. The organic layer was separated and concentrated in vacuo. The crude product was purified by column chromatography on silica gel eluting with dichloromethane:methanol:ammonia 100:10:1 to yield the title product (210 mg, 27%), mixture of diastereoisomers.

¹HNMR (CD3OD, 400 MHz) δ: 1.12-1.18 (m, 2H), 1.46-1.54 (m, 4H), 1.67-1.77 (m, 2H), 2.04 (m, 0.5H), 2.16 (m, 1H), 2.26 (m, 2H), 2.48 (m, 0.5H), 2.95 (m, 1H), 3.25 (m, 1H), 3.53-3.57 (m, 2H), 3.78-3.86 (m, 4H), 4.12 (m, 1H), 7.05 (m, 1H), 7.11-7.22 (m, 2H); MS APCI⁺ m/z 349 [MH]⁺.

### EXAMPLE 3

### N-Bicyclo[2.2.1]hept-2-yl-2-(trifluoromethyl)N-[(3S)pyrrolidin-3-yl]benzamide

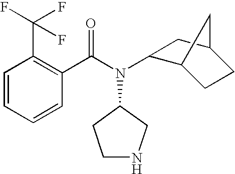

N-Bicyclo[2.2.1]hept-2-yl-2-(trifluoromethyl)N-[(3S)-pyrrolidin-3-yl]benzamide was prepared from the compound of preparation 6 by a method similar to that described for example 2 to yield the title product, 300 mg (24%) as a mixture of diastereoisomers.

¹HNMR (CD3OD, 400 MHz) δ: 1.04 (m, 1H), 1.09 (m, 1H), 1.15 (m, 1H), 1.50 (m, 3H), 1.70-1.84 (m, 3H), 2.12-2.24 (m, 3.5H), 2.43 (m, 0.5H), 2.78 (m, 0.5H), 2.93 (m, 0.5H), 3.04 (m, 1H), 3.14 (m, 0.5H), 3.40-3.49 (m, 1H), 3.62 (m, 0.5H), 3.99 (m, 1H), 7.45 (m, 1H), 7.62 (m, 1H), 7.72-7.76 (m, 2H); MS APCI⁺ m/z 353 [MH]⁺.

### EXAMPLE 4

### N-Bicyclo[2.2.1]hept-2-yl-2-chloro-N-[(3S)-pyrrolidin-3-yl]benzamide

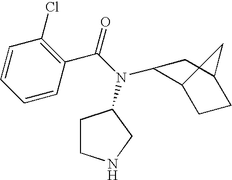

N-Bicyclo[2.2.1]hept-2-yl-2-chloro-N-[(3S)-pyrrolidin-3-yl]benzamide was prepared from the compound of preparation 7 by a method similar to that described for example 2 to yield the title product, 640 mg (90%) as a mixture of diastereoisomers.

¹HNMR (CD3OD, 400 MHz) δ: 1.10 (m, 1H), 1.17 (m, 1H), 1.44-1.51 (m, 3H), 1.65-1.68 (m, 1.5H), 1.79-1.86 (m, 1.5H), 2.06 (m, 0.5H), 2.16-2.21 (m, 1.5H), 2.26 (m, 1.5H), 2.46 (m, 0.5H), 2.78 (m, 0.5H), 2.94 (m, 0.5H), 3.09 (m, 1H), 3.20 (m, 0.5H), 3.42 (m, 1H), 3.50 (m, 0.5H), 3.74 (m, 1H), 4.04 (m, 1H), 7.33 (m, 0.5H), 7.38-7.42 (m, 2.5H), 7.47 (m, 1H); MS APCI⁺ m/z 319 [MH]⁺.

### EXAMPLE 5

### 2,3-Dichloro-N-[(3S)-pyrrolidin-3-yl]-N-(3,3,3-trifluoropropyl)benzamide hydrochloride

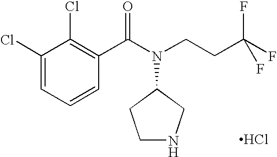

A solution of 4M hydrochloric acid in dioxane (4 ml) was added to the Boc protected compound of Preparation 10 (400 mg, 0.88 mmol). The reaction mixture was stirred at room temperature for 30 minutes and then concentrated in vacuo. The residue was azeotroped with diethyl ether, evaporated and dried to yield the title product as a white foam (250 mg, 72%).

¹HNMR (CD3OD, 400 MHz) δ: 2.47-2.59 (m, 4H), 3.26 (m, 1H), 3.43 (m, 1H), 3.51-3.58 (m, 2H), 3.75-3.81 (m, 2H), 4.35 (m, 1H), 7.38 (m, 1H), 7.40 (t, 1H), 7.68 (d, 1H); MS APCI⁺ m/z 355 [MH]⁺

### EXAMPLE 6

### 2-Phenoxy-N-[(3S)-pyrrolidin-3-yl]-N-(3,3,3-trifluoropropyl)benzamide hydrochloride

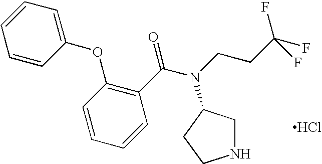

The title compound was prepared from the compound of preparation 11 by a method similar to that described for example 5 to yield the title product (251 mg, 99%) as a white solid.

¹HNMR (CD3OD, 400 MHz) δ: 2.05 (m, 1H), 2.43 (m, 1H), 2.52-2.59 (m, 2H), 3.14-3.23 (m, 2H), 3.39-3.74 (m, 4H), 4.31 (m, 1H), 6.97-7.03 (m, 3H), 7.15 (m, 1H), 7.27 (m, 1H), 7.35-7.39 (m, 2H), 7.43-7.49 (m, 2H); MS APCI⁺ m/z 379 [MH]⁺.

### EXAMPLE 7

### N-(2,2-Difluoropropyl)-2-phenoxy-N-[(3S)-pyrrolidin-3-yl]benzamide hydrochloride

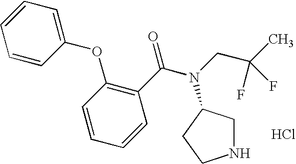

The title compound was prepared as a white foam (639 mg, quantitative) from the compound of preparation 14, following the procedure described for example 5.

¹HNMR (CD3OD, 400 MHz, rotamers) δ: 1.42-1.57 (m, 3H), 1.93-2.58 (m, 2H), 3.08-3.77 (m, 4H), 3.80-3.94 (m, 2H), 4.38 (m, 1H), 6.95-7.01 (m, 3H), 7.12-7.26 (m, 2H), 7.33-7.50 (m, 4H); MS APCI⁺ m/z 361 [MH]⁻.

### EXAMPLE 8

### 2-phenoxy-N-[(3S)-pyrrolidin-3-yl]-N-(2,2,2-trifluoroethyl)benzamide hydrochloride

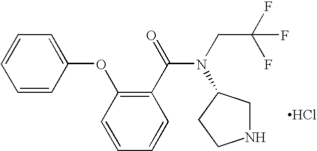

The Boc protected compound of Preparation 19 (1.00 g, 2.15 mmol) was dissolved in methylene chloride (10 ml) and a solution of 4M hydrochloric acid in dioxane (10.8 ml, 43.00 mmol, 20 eq.) was added. The reaction mixture was stirred at room temperature for 3 hours, after which time the volatiles were evaporated under reduced pressure, affording 749 mg of the title compound as a white solid (83%).

¹HNMR (CD₃OD, 400 MHz) δ: 1.91-2.61 (b, 2H), 3.12 (b, 2H), 3.34-3.63 (m, 1H), 3.73 (m, 1H), 4.20 (m, 2H), 4.38 (b, 1H), 6.99 (t, 3H), 7.17 (t, 1H), 7.28 (t, 1H), 7.34-7.52 (m, 4H); MS APCI⁺ m/z 365 [MH]⁺

### EXAMPLE 9

### N-(2,2-difluoroethyl)-2-phenoxy-N-[(3S)-pyrrolidin-3-yl]benzamide hydrochloride

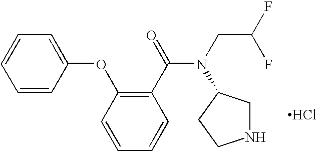

The Boc protected compound of Preparation 20 (730 m g, 1.63 mmol) was dissolved in methylene chloride (10 ml) and then a solution of 4M hydrochloric acid in dioxane (8.2 ml, 32.6 mmol, 20 eq.) was added. The solution was stirred at room temperature for 3 hours, after which time the volatiles were evaporated under reduced pressure affording 554 mg of the title compound as a yellow gum (85%).

¹HNMR (CD₃OD, 400 MHz) δ: 1.93-2.60 (b, 2H), 3.15 (b, 2H), 3.34-3.63 (m, 1H), 3.67-3.95 (m, 3H), 4.36 (b, 1H), 5.99 (t, 1H), 6.93-7.05 (m, 3H), 7.17 (t, 1H), 7.26 (t, 1H), 7.33-7.52 (m, 4H); MS APCI⁺ m/z 347 [MH]⁺

### EXAMPLE 10

### 2,3-dichloro-N-(dicyclopropylmethyl)-N-[(3S)-pyrrolidin-3-yl]benzamide hydrochloride

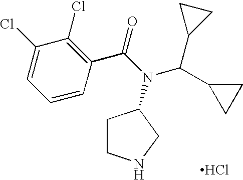

50 mg (0.11 mmol) of the compound of preparation 22 were dissolved in 3 ml of a solution of 4M hydrochloric acid in dioxane. The solution was stirred at room temperature for 3 hours, after which time the volatiles were removed under reduced pressure affording 42 mg of the title compound as a white solid (100%).

¹H-NMR (400 MHz, CD₃OD) δ: 0.31 (m, 2H), 0.41 (m, 2H), 0.55 (m, 2H), 0.68 (m, 2H), 1.04-1.17 (m, 2H), 2.52-2.69 (m, 3H), 3.62 (m, 2H), 3.87 (m, 2H), 4.76 (m, 1H), 7.35 (quartet, 1H), 7.45 (m, 1H), 7.68 (d, 1H); MS m/z APC+: 353 [MH]⁺

### EXAMPLE 11

### N-(2-methoxyethyl)-2-phenoxy-N-[(3S)-pyrrolidin-3-yl]benzamide hydrochloride

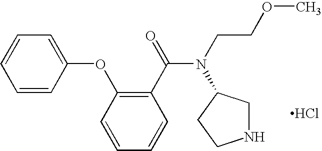

The title compound was prepared by a similar method to that described for example 10 using the protected amine of preparation 25 to produce the desired product, 354 mg (87%) as a white gum.

¹HNMR (CD₃OD, 400 MHz) δ: 2.00-2.55 (m, 2H), 3.07-3.80 (m, 1H), 4.32 (m, 1H), 6.99 (m, 3H), 7.12 (t, 1H), 7.22 (t, 1H), 7.41 (m, 4H); MS APCI⁺ m/z 341 [MH]⁺

### EXAMPLE 12

All of the following compounds exhibited an NRI Ki and/or an SRI Ki of less than 200 nM when tested using the biological activity assay described:
N-bicyclo[2.2.1]hept-2-yl-4-chloro-2-methoxy-N-[(3S)-pyrrolidin-3-yl]benzamide,
N-bicyclo[2.2.1]hept-2-yl-2-chloro-N-[(3S)pyrrolidin-3-yl]benzamide,
N-[(1R,5S)-bicyclo[3.2.0]hept-3-yl]-2,3-dichloro-N-[(3S)-pyrrolidin-3-yl]benzamide,
2,3-dichloro-N-(dicyclopropylmethyl)N-[(3S)-pyrrolidin-3-yl]benzamide,
2,3-dichloro-N-[(3S)pyrrolidin-3-yl]-N-(3,3,3-trifluoropropyl)benzamide,
N-bicyclo[2.2.1]hept-2-yl-N-[(3S)pyrrolidin-3-yl]-2-(trifluoromethyl)benzamide,
2-phenoxy-N-[(3S)-pyrrolidin-3-yl]-N-(3,3,3-trifluoropropyl)benzamide,
2-phenoxy-N-[(3S)-pyrrolidin-3-yl]-N-(2,2,2-trifluoroethyl)benzamide,
N-(2,2-difluoropropyl)-2-phenoxy-N-[(3S)-pyrrolidin-3-yl]benzamide,
N-(2,2-difluoroethyl)-2-phenoxy-N-[(3S)-pyrrolidin-3-yl]benzamide,
N-(2-methoxyethyl)-2-phenoxy-N-[(3S)-pyrrolidin-3-yl]benzamide,
2,4-dichloro-N-[(3S)-pyrrolidin-3-yl]-N-(3,3,3-trifluoropropyl)benzamide (MS m/z APCI+: 355 [MH]⁺),
3,4-dichloro-N-[(3S)-pyrrolidin-3-yl]-N-(3,3,3-trifluoropropyl)benzamide (MS m/z APCI+: 355 [MH]⁺),
2,3-dichloro-N-[(3S)-pyrrolidin-3-yl]-N-(2,2,2-trifluoroethyl)benzamide (MS m/z APCI+: 341 [MH]⁺), 2-(ethylthio)N-[(3S)-pyrrolidin-3-yl]-N-(3,3,3-difluoropropyl)benzamide (MS m/z APCI+: 347 [MH]⁺),
N-[(3S)pyrrolidin-3-yl]-2-(trifluoromethoxy)-N-(3,3,3-trifluoropropyl)benzamide (MS APCI+ m/z: 371 [MH]⁺).

### Biological Activity

The compounds were tested for biological activity by their ability to inhibit binding of selective radioligands at the human serotonin and noradrenaline transporters (SERT and NET, respectively), using scintillation proximity assay (SPA) technology. The SPA binding was performed using cellular membrane preparations prepared from cell lines expressing human cDNA encoding either SERT or NET (hSERT, hNET), using the radioligands ³H-citalopram and ³H-nisoxetine.

i) Cell Culture Methodology

Human embryonic kidney cells (HEK-293) expressing each transporter were maintained as a continuous culture, using standard cell culture techniques, in 50 mL of growth medium (see Media and Buffers for composition) in 225 cm² flasks, at 37° C. in a humidified atmosphere with 5% CO₂ present. Cells were passaged from a 90% confluent monolayer at a ratio of 1:3-1:4.

For cell harvesting, the growth medium was removed from the monolayer and the cells were incubated with cell dissociation solution (Sigma) until showing signs of dissociation. The cells were subsequently knocked from the base of the flask and pelleted by centrifugation for storage (frozen at −80° C.) prior to further use.

ii) Cellular Membrane Preparation

Cell pellets were thawed on ice and resuspended in 3 mL of membrane preparation buffer (see Media and Buffers for composition) per 1 mL of packed cell volume, using a vortex mixer to disperse the cell pellet.

After incubation on ice for 10 minutes, the suspension was homogenised for four individual 10 second intervals using a hand-held homogeniser. The homogenate was then centrifuged at 1075×g for 20 minutes at 4° C.

The supernatants were then collected and retained. Initial cell & nuclei pellets (P1) were subsequently rehomogenised and centrifuged using the conditions cited above, and the supernatants collected and pooled with those retained from the first spin.

The pooled supernatants were centrifuged at 35000×g for 30 minutes at 4° C., and the supernatants discarded. The pellets (P2) were then resuspended in 1 mL of membrane preparation buffer per 1 mL of the original packed cell volume. Protein concentrations were then measured and the membrane suspension was finally frozen in aliquots of set volume and stored at −80° C. prior to use in assays.

iii) Assay Methodology

### A. Determination of Optimal Assay Conditions for Individual Membrane Batches

The specific SPA bead type differed for each transporter, wheat germ agglutinin-coated yttrium silicate (YSi WGA) SPA beads were used for hSERT and WGA-coated polyvinyltoluene (PVT WGA) SPA beads for hNET assays. For each batch of membrane used, optimal concentrations of bead and membrane were determined

Tritiated radioligands specific to each transporter (³H-citalopram for hSERT and ³H-nisoxetine for hNET) were used. The assay free radioligand concentration was expressed as a percentage of the total free radioligand concentration to give an estimate of the radioligand depletion. The radioligand depletion in assays for both transporters was less than 30% to ensure that there was sufficient radioligand available for binding. The ligand depletion value was also used for selecting the optimal assay conditions when using new batches of membranes.

The affinity of the specific radioligand for the respective transporter was determined for each membrane batch at the selected protein and bead concentrations. This was achieved by the determination of the K_{D}, the concentration of free radioligand at which 50% of the transporter binding sites were occupied. The mean K_{D} for a radioligand at a batch of membranes was determined from data from a minimum of three separate assays. The mean K_{D} was subsequently used for all assays using the membrane batch profiled to enable determination of Kᵢ values of compounds studied using the method determined by Cheng and Prussoff (Cheng Y C and Prusoff W H. Relationship between the inhibition constant (Ki) and the concentration of inhibitor which causes 50% inhibition of an enzymatic reaction. Biochem Pharmacol 1973; 22:2099-3108.)

### B. Assay Protocol

### Bead/Membrane Complex Preparation

The required amount of membrane was thawed on ice and added to a predetermined volume of bead suspension in assay buffer. The beads were then pre-coupled by incubating the predetermined protein quantity per mg of bead on a shaker at a temperature of 4° C. for 2 hours. Subsequently, the bead/membrane complex was spun down at 865×g for 5 minutes. The resulting pellet was resuspended in assay buffer and this spin/wash step then repeated. The final pellet was then resuspended in assay buffer at the specific concentration required for the final assay.

### Ligand Preparation

An aliquot of [³H]-radioligand stock was diluted in assay buffer to give a pre-determined final assay concentration less than the equilibrium dissociation constant (K_{D}) value.

### Compound Plate Preparation

All test compounds were prepared at a concentration of 4 mM in 100% dimethyl sulphoxide (DMSO) from dry samples. Compounds were diluted in 0.75% DMSO in ddH₂O to give appropriate test concentrations in a 384 well plate to give a final volume of 20 μL.

The same volume of assay buffer was added to specific wells of the plate to enable subsequent measurement of total radioligand binding. Furthermore, 20 μL a high concentration of compound specific to each transporter assay was subsequently added to predetermined wells to determine non-specific binding (NSB). Fluoxetine (10 μM final assay concentration) was used for hSERT and desipramine (40 μM final assay concentration) for hNET.

For each individual transporter assay, 20 μL of the prepared specific radioligand was added to each well of the final assay plates (containing compound solutions). Subsequently, 20 μL of the corresponding bead/membrane complex was added to each well of the final assay plate, ensuring that the suspension was mixed well. The plates were then sealed and incubated, with shaking, for 1 hour at room temperature. The plates were subsequently incubated for an additional 6 hours, with dark adaptation, prior to reading.

### C. Data Analysis

The assay window (specific binding) per plate was calculated by subtracting the mean NSB readings (in counts per minute, or cpm) from the mean of total binding readings. Subsequently the cpm read per well (with mean NSB subtracted) were expressed as a percentage of the plate window to determine the amount of radioligand bound to the transporter.

These values were plotted against the concentration of the compound tested and a sigmoidal inhibitory concentration effect curve was fitted to the data using a four-parameter logisitic equation and free-fitting parameters to give an IC₅₀ value (the concentration of compound required to inhibit 50% of the specific binding at the neurotransmitter transporter).

The inhibitory dissociation constant (Kᵢ) value was then calculated from the IC₅₀ value using the Cheng-Prusoff equation

Following determination of individual Kᵢ values for compounds tested, an overall geometric mean was calculated together with 95% confidence intervals and n values, where n is the total number of individual Kᵢ values. The resulting Kᵢ data of Examples 1, 3, 4 and 5 can be seen in Table 1.

iv) Media and Buffers

hSERT Cell Growth Medium
DMEM, 10% (w/v) dialysed FCS
2 mM L-glutamine (diluted from 200 mM stock)
25 mM HEPES (diluted from 1 M stock)
250 μg/mL genetecin
hNET Cell Growth Medium
DMEM, 10% (w/v) FCS
2 mM L-glutamine (diluted from 200 mM stock)
25 mM HEPES (diluted from 1 M stock)
250 μg/mL genetecin

### Membrane Preparation Buffer

20 mM HEPES (diluted from 1M stock with ddH₂O), pH 7.4 at room temperature, stored at 4° C. Prior to use, one complete protease inhibitor tablet was dissolved per 50 mL of buffer.

### Assay Buffer (1.5× Final Assay Concentration)

30 mM HEPES (diluted from 1 M stock with ddH₂O) and 180 mM NaCl (diluted from 5 M stock with ddH₂O), pH 7.4 at room temperature, stored at 4° C.

The NRI Ki and the SRI Ki of the compounds of Examples 1 to 12 were determined as follows. A selection of the results are set out below in Table 1. All of the Example compounds exhibited an NRI Ki and/or an SRI Ki of less than 200 nM.

TABLE 1

| Compound | SRI Ki (nM) | NRI Ki (nM) |
|---|---|---|
| Example 1 | 9 | 12 |
| Example 3 | 30 | 28 |
| Example 4 | 6 | 25 |
| Example 5 | 21 | 58 |
| Example 7 | 1590 | 10 |
| Example 10 | 11 | 72 |

The compounds can also be tested in specific disease models, such as the pain models as follows:

### Neuropathic Pain

The activity of a compound in the treatment of neuropathic pain may be measured according to the following test protocol.

Animals: Male Sprague Dawley rats are housed in appropriately sized groups. All animals are kept under a 12 h light/dark cycle (lights on at 07 h 00 min) with food and water ad libitum. All experiments are carried out by an observer blind to the treatments and in accordance with the Home Office Animals (Scientific Procedures) Act 1986.

### Chronic Constriction Injury (CCI) Rat Model of Neuropathic Pain

The CCI of sciatic nerve is performed as previously described by Bennett and Xie (Bennett G J, Xie Y K. A peripheral mononeuropathy in rat that produces disorders of pain sensation like those seen in man. Pain: 33:87-107, 1988). Animals are anaesthetised with a 2% isofluorane/O2 mixture. The right hind thigh is shaved and swabbed with 1% iodine. Animals are then transferred to a homeothermic blanket for the duration of the procedure and anaesthesia maintained during surgery via a nose cone. The skin is cut along the line of the thighbone. The common sciatic nerve is exposed at the middle of the thigh by blunt dissection through biceps femoris. About 7 mm of nerve is freed proximal to the sciatic trifurcation, by inserting forceps under the nerve and the nerve gently lifted out of the thigh. Suture is pulled under the nerve using forceps and tied in a simple knot until slight resistance is felt and then double knotted. The procedure is repeated until 4 ligatures (4-0 silk) are tied loosely around the nerve with approx 1 mm spacing. The incision is dosed in layers and the wound treated with topical antibiotics.

### Streptozocin (STZ)-Induced Diabetes Neuropathy in the Rat

Diabetes is induced by a single intraperitoneal injection of streptozotocin (50 mg/kg) freshly dissolved in 0.9% sterile saline. Streptozotocin injection induces a reproducible mechanical allodynia within 3 weeks, lasting for at least 7 weeks (Chen and Pan, (Chen S R and Pan H L. Hypersensitivity of Spinothalamic Tract Neurons Associated With Diabetic Neuropathic Pain in Rats. J Neurophysiol 87: 2726-2733, 2002).

### Assessment of Static and Dynamic Allodynia

### Static Allodynia.

Animals are habituated to wire bottom test cages prior to the assessment of allodynia. Static allodynia is evaluated by application of von Frey hairs (Stoelting, Wood Dale, Ill., USA.) in ascending order of force (0.6, 1, 1.4, 2, 4, 6, 8, 10, 15 and 26 grams) to the plantar surface of hind paws. Each von Frey hair is applied to the paw for a maximum of 6 sec, or until a withdrawal response occurred. Once a withdrawal response to a von Frey hair is established, the paw is re-tested, starting with the filament below the one that produced a withdrawal, and subsequently with the remaining filaments in descending force sequence until no withdrawal occurs. The highest force of 26 g lifts the paw as well as eliciting a response, thus represented the cut off point. Each animal has both hind paws tested in this manner. The lowest amount of force required to elicit a response is recorded as paw withdrawal threshold (PWT) in grams. Static allodynia is defined as present if animals respond to a stimulus of, or less than, 4 g, which is innocuous in naive rats (Field M J, Bramwell S, Hughes J, Singh L. Detection of static and dynamic components of mechanical allodynia in rat models of neuropathic pain: are they signalled by distinct primary sensory neurones? Pain,1999; 83:303-11).

### Dynamic Allodynia

Dynamic allodynia is assessed by lightly stroking the plantar surface of the hind paw with a cotton bud. To avoid recording general motor activity, care is taken to perform this procedure in fully habituated rats that are not active. At least two measurements are taken at each time point, the mean of which represents the paw withdrawal latency (PWL). If no reaction is exhibited within 15 sec the procedure is terminated and animals are assigned this withdrawal time. A pain withdrawal response is often accompanied with repeated flinching or licking of the paw. Dynamic allodynia is considered to be present if animals respond to the cotton stimulus within 8 sec of commencing stroking (Field et al, 1999).

### Nociceptive Pain

The activity of a compound in the treatment of nociceptive pain may be measured according to the following test protocols.

### Hotplate

Experimental Procedure: Male Sprague Dawley rats are placed on a hot plate (Ugo Basile, Italy) maintained at 55±5° C. The time between placement of the animal on the hot plate and occurrence of either licking of fore or hind paw, shaking or jumping off the surface is measured. Baseline measurements are made and animals reassessed following drug administration. The cut off time for hot plate latencies is set at 20 seconds to prevent tissue damage.

### Ovariohysterectomy (OVX)

Experimental Procedure: Female Sprague Dawley rats are placed into an anaesthetic chamber and anaesthetised with a 2% isofluorane O₂ mixture. During surgery, anaesthesia is maintained via a nose cone. OVX is performed via a midline incision (2 cm in length) in the linea alba, whilst the animal is on a heat blanket. The ovarian ligaments and cervix are ligated with 5-0 silk, using a single clamp technique. The ovaries and uterus are then removed. The abdominal wall is closed using 4 simple interrupted sutures and the skin closed using 4 wound clips. Immediately after surgery animals are placed in individual plexiglass chambers. Once the animal has recovered from the anaesthetic the abdominal body postures are recorded in 30 min bins at various time points. Postures scored are humpback position, contraction of the muscle of the abdomen associated with inward movements of the hind limb, stretching of the body and squashing of the lower abdomen against the floor. Each of these behaviours is scored as one posture.

### Brennan

Experimental Procedure: Male Sprague Dawley rats are placed into an anaesthetic chamber and anaesthetised with a 2% isofluorane O₂ mixture. During surgery, anaesthesia is maintained via a nose cone. The plantar aspect of the right hind paw is cleaned with 50% ethanol. A 1 cm long longitudinal incision is made with a number 11 blade through the skin and fascia of the plantar aspect of the foot, starting 0.5 cm from the proximal edge of the heel and extending toward the toes. The plantaris muscle is elevated using forceps and incised longitudinally, the muscle origin and insertion remain intact. After haemostasis with gentle pressure, the skin is dosed with two simple sutures of braided silk.

### Mono-Iodoacetate (MIA)-Induced OA Model

Male 6 weeks-old Sprague-Dawley (SD, Japan SLC or Charles River Japan) rats are anesthetized with pentobarbital. Injection site is shaved and cleaned with 70% ethanol. 25 μl of MIA solution or saline is injected in the right knee joint using a 29G needle. 7, 14, 19 and 20 days after the MIA injection, train rats to measure the weight bearing (WB) without their stress. 21 days after the MIA injection, the WB on two of each hind paw is measured and the WB deficit is calculated. Define the WB deficit value as “pre value”. Arrange for experimental group evenly in consideration of pre value and prepre value. After the administration of test compounds or vehicle, the WB on two of each hind paw was measured.

### Cancer Pain Model

These experiments use adult male C3H/HeN mice (Nihon SLC, Shizuoka, Japan). The mice are housed in accordance with National Institutes of Health guidelines in a vivarium maintained at 22° C. with a 12-hour alternating light-dark cycle, and were given food and water ad libitum. The sarcoma injection protocol which is used has been described. After induction of general anesthesia with an inhalation of isofluran (2%), a superficial incision is made in the skin overlying the patella, using Mora scissors. The patellar ligament is then cut, exposing the condyles of the distal femur. A 30-gauge needle is inserted at the level of the intercondylar notch and into the medullary canal to create an initial core pathway. After the initial core is made, a 29-gauge needle is used to make the final pathway into the bone. A 0.5-mm depression is then made using a half-round bur in a pneumatic dental high speed handpiece, to serve as mechanical retention for the dental resin plug. Then, 20 μl α-minimum essential media (Sigma; sham injection) or 20 μl media containing 1×10⁵ 2472 osteolytic sarcoma cells (American Type Culture Collection, Rockville, Md.; sarcoma injection) is injected using a 29-gauge needle and a 0.25 cc syringe. To prevent leakage of cells outside the bone, the injection site is closed with dental resin, followed by copious irrigation with filtered water. Wound closure is achieved using auto wound clips (Becton Dickinson, San Jose, Calif.). Wound clips are removed at day 5 to prevent interference with behavioral testing.

### Assessment of Static and Dynamic Allodynia

### Static Allodynia.

Animals are habituated to wire bottom test cages prior to the assessment of allodynia. Static allodynia is evaluated by application of von Frey hairs (Stoelting, Wood Dale, Ill., USA.) in ascending order of force (0.6, 1, 1.4, 2, 4, 6, 8, 10, 15 and 26 grams) to the plantar surface of hind paws. Each von Frey hair is applied to the paw for a maximum of 6 sec, or until a withdrawal response occurs. Once a withdrawal response to a von Frey hair is established, the paw is re-tested, starting with the filament below the one that produces a withdrawal, and subsequently with the remaining filaments in descending force sequence until no withdrawal occurs. The highest force of 26 g lifts the paw as well as eliciting a response, thus represents the cut off point. Each animal has both hind paws tested in this manner. The lowest amount of force required to elicit a response is recorded as paw withdrawal threshold (PWT) in grams. Static allodynia is defined as present if animals respond to a stimulus of, or less than, 4 g, which is innocuous in naive rats (Field M J, Bramwell S, Hughes J, Singh L. Detection of static and dynamic components of mechanical allodynia in rat models of neuropathic pain: are they signalled by distinct primary sensory neurones? Pain,1999; 83:303-11).

### Dynamic Allodynia

Dynamic allodynia is assessed by lightly stroking the plantar surface of the hind paw with a cotton bud. To avoid recording general motor activity, care is taken to perform this procedure in fully habituated rats that are not active. At least two measurements are taken at each time point, the mean of which represents the paw withdrawal latency (PWL). If no reaction is exhibited within 15 sec the procedure is terminated and animals are assigned this withdrawal time. A pain withdrawal response is often accompanied with repeated flinching or licking of the paw. Dynamic allodynia is considered to be present if animals respond to the cotton stimulus within 8 sec of commencing stroking (Field et al, 1999).

### Radiant Heat Paw Withdrawal

Experimental procedure: Thermal paw withdrawal is assessed using the rat plantar test (Ugo Basile, Italy) following a modified method of Hargreaves et al., 1988. Rats are habituated to the apparatus that consists of three individual perspex boxes on an elevated glass table. A mobile radiant heat source is located under the table and focused onto the hind paw and paw withdrawal latencies (PWL) are recorded. There is an automatic cut off point of 22.5 s to prevent tissue damage. PWL are taken 2-3 times for both hind paws of each animal, the mean of which represents baselines for right and left hind paws. The apparatus is calibrated to give a PWL of approximately 10 s.

### Weight Bearing

Experimental procedure: Animals are examined for hypersensitivity in the weight-bearing test, using an “incapacitance tester” (Linton Instruments, Diss, Norfolk, U.K.). Rats were positioned with their fore limbs up on a perspex slope and hind limb weight distribution was measured via force transducers under each of the hind paws. Each animal is placed in the apparatus and the weight load exerted by the hind paws is noted. The difference in weight bearing is calculated by subtracting the ipsilateral (injured) paw from the contralateral paw (normal) and this constitutes the raw data.

### Inflammatory Pain

The activity of compound in the treatment of inflammatory pain may be measured according to the following test protocol.

### CFA-Induced Weight Bearing Deficits in Rats

Male 7-week-old SD rats are fasted overnight. CFA (360 μg of Mycobacterium Tuberculosis H37 RA (Difco Laboratories) in 100 μL of liquid paraffin (Wako)) is injected into the rat's right hind footpad. Two days after the administration of CFA, the changes in hind paw weight distribution between the left (ipsilateral) and the right (contralateral) limbs are measured as an index of pain by using Linton Incapacitance tester (Linton Instrumentation, UK). The test compound suspended in 0.1% MC (Wako) is administered orally in a volume of 0.1 mL per 100 g body weight. Each animal is placed in the apparatus and the weight load exerted by the hind paws is measured before, 1, 2 and 4 hours after drug administration.

### Carrageenin-Induced Mechanical Hyperalgesia in Rats

Male 4-week-old SD rats are fasted overnight. Hyperalgesia is induced by intraplantar injection of Lambda-carrageenin (0.1 ml of 1% w/v solution in saline, Zushikagaku). The test compound (1 ml of 0.1% methylcellulose/100 g body weight) is given orally at 5.5 hours after the carrageenin injection. The paw withdrawal threshold (gram) is measured by analgesimeter (Ugo Basile) at 3.5, 4.5, 6.5 and 7.5 hours after the carrageenin injection. (Randall L. O. & Selitto I. J., Arch. Int. Pharmacodyn. 111, 409-419, 1957)

### Carrageenan-Induced Thermal Hyperalgesia (CITH) in the Rat

Thermal hyperalgesia is assessed using the rat plantar test (Ugo Basile, Comerio, Italy), according to a method modified by Hargreaves et al. (1988). Briefly, rats are habituated to the apparatus that consists of three individual Perspex boxes on a glass table. A mobile radiant heat source is located under the table and focused onto the desired paw. Paw withdrawal latencies (PWLs) are recorded three times for both hind paws of each animal, the mean of which represents baseline for left and right hind paws. The apparatus is calibrated to give a PWL of approximately 10 s in naïve rats. To prevent tissue damage of the plantar zone, a 22.5 sec cut-off is observed. Lambda carrageenan is injected intraplantarly (100 μl, 20 mg/ml) the right hind paw and baseline recordings of PWT are taken 2 hr post administration.

### Visceral Pain

The activity of a compound in the treatment of visceral pain may be measured according to the following test protocols.

Several models are available to determine if a compound is effective in treating disorders of the viscera. These models include a LPS model (Eutamene H et al, J Pharmacol Exp Ther 2000 295 (1):162-7), a TNBS model (Diop L. et al, Gastroenterology 1999, 116, 4(2): A986), a IBD model (Clemett D, Markham A, Drugs 2000 April; 59(4):929-56), a pancreatic pain model (Isla A M, Hosp Med 2000 June; 61(6):386-9) and a visceral non digestive pain model (Boucher M et al, J Urol 2000 July; 164(1):203-8).

### TNBS-Induced Chronic Visceral Allodynia in Rats

In this experimental model of colonic distension in awake rats, previous injection of trinitrobenzenesulfonic acid (TNBS) into the proximal colon lowered the visceral pain threshold.

Materials and methods: Male Sprague-Dawley rats are used. The animals are housed 3 per cage in a regulated environment (20±1° C., 50±5% humidity, with light 8:00 am to 8:00 pm). At day 0, under anesthesia (ketamine 80 mg/kg i.p.; acepromazine 12 mg/kg i.p.), the injection of TNBS (50 mg/kg in ethanol 30%), or saline (1.5 ml/kg) for control rats, is performed into the proximal colon wall (1 cm from the cecum). After the surgery, animals are individually housed in polypropylene cages and kept in a regulated environment (20±1° C., 50±5% humidity, with light 8:00 a.m. to 8:00 p.m.) during 7 days. At day 7 after TNBS administration, a balloon (5-6 cm length) is inserted by anus, and kept in position (tip of balloon 5 cm from the anus) by taping the catheter to the base of the tail. Oral administration of the test compound is performed 1 h before the colonic distension cycle: the balloon is progressively inflated by steps of 5 mm Hg (0.667 kPa), from 0 to 75 mm Hg, each step of inflation lasting 30 s. Each cycle of colonic distension is controlled by a standard barostat. The threshold (mm Hg) corresponds to the pressure which produced the first abdominal contraction, and the cycle of distension is then discontinued. The colonic threshold is determined after performance of four cycles of distension on the same animal.

### LPS-Induced Rectal Hypersensitivity in Rats

Intraperitoneal injection of bacterial lipo-polysaccharide (LPS) has been shown to induce rectal hyperalgesia in awake rats.

Materials and methods: Animals are surgically prepared for electromyography: rats are anaesthetized by intraperitoneal injection of acepromazine (0.6 mg/kg) and ketamine (120 mg/kg). Three groups of three electrodes are implanted in the abdominal external oblique musculature, just superior to the inguinal ligament. Electrodes are exteriorized on the back of the neck and protected by a glass tube attached to the skin. Animals are individually housed in polypropylene cages and kept in a temperature-controlled room (21° C.). Food (UAR pellets, Epinay, France) and water are provided ad libitum.

Electromyographic recordings begin five days after surgery. The electrical activity of abdominal striated muscles is recorded with an electroencephalograph machine (Mini VIII Alvar, Paris, France) using a short time constant (0.03 s) to remove low-frequency signals (<3 Hz) and a paper speed of 3.6 cm/min. Spike bursts are recorded as an index of abdominal contractions.

Distension procedure: Rats are placed in plastic tunnels (6 cm diameter×25 cm long), where they cannot move, escape, or turn around, in order to prevent damage to the balloon. Animals are accustomed to this procedure for four days before rectal distension in order to minimize stress reactions during experiments. The balloon used for distension is an arterial embolectomy catheter (Fogarty, Edwards Laboratories Inc.). Rectal distension is performed by insertion of the balloon (2 mm diameter×2 cm long) into the rectum, at 1 cm from the anus, and catheter is fixed at the base of the tail. It is inflated progressively with tepid water by steps of 0.4 ml, from 0 to 1.2 ml, each step of inflation lasting 5 min. To detect possible leakage, the volume of water introduced in the balloon is checked by complete removal with a syringe at the end of the distension period.

1-23. (canceled)

24. A compound of Formula (I) 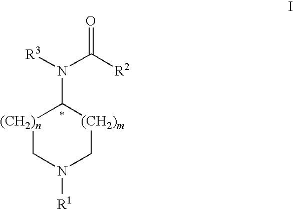
and pharmaceutically and/or veterinarily acceptable derivatives thereof, wherein:
R¹ is H, C₁₋₆alkyl, —C(A)D, C₃₋₈cycloalkyl, aryl, het, aryl-C₁₋₄alkyl or het-C₁₋₄alkyl, wherein the cycloalkyl, aryl or het groups are optionally substituted by at least one substituent independently selected from C₁₋₈allyl, C₁₋₈alkoxy, OH, halo, CF₃, OCHF₂, OCF3, SCF₃, hydroxy-C₁₋₆alkyl, C₁₋₄alkoxy-C₁₋₆alkyl and C₁₋₄alkyl-S—C₁₋₄alkyl;
A is S or O;
D is H, C₁₋₆alkyl, aryl, het, aryl-C₁₋₄alkyl or het-C₁₋₄alkyl;
aryl represents phenyl, naphthyl, anthracyl or phenanthryl;
het represents an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom;
R² is aryl¹ or het¹, each optionally substituted by at least one substituent independently selected from B;
B represents C₁₋₈alkyl, C₁₋₈alkoxy, aryl 2, het², Oaryl², Ohet², SC₁₋₆alkyl, Saryl², Shet², OH, halo, CF₃, CHF₂, OCHF₂, OCF₃, SCF₃₁ CF₂CF₃, CH₂CF₃, CF₂CH₃, hydroxy-C₁₋₆alkyl, C₃₋₆cycloalkyl, C₃₋₆cycloalkyl-C₁₋₄alkyl, C₃₋₆cycloalkyl-C₁₋₄alkoxy, C₃₋₆cycloalkyl-O—C₁₋₄alkyl, C₃₋₆cycloalkyl-C₁₋₄alkoxy-C₁₋₄alkyl, OC₃₋₆cycloalkyl, SC₃₋₆cycloalkyl, C₁₋₄alkoxy-C₁₋₆alkyl, aryl²-C₁₋₄alkyl and C₁₋₄alkyl-S—C₁₋₄alkyl, wherein the aryl² and het² groups are optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃;
aryl¹ represents phenyl, naphthyl, anthracyl or phenanthryl;
het¹ is an aromatic 5- or 6-membered heterocycle which contains at least one N, O or S heteroatom, optionally fused to an aryl group;
at each occurrence aryl² independently represents phenyl, naphthyl, anthracyl or phenanthryl;
at each occurrence het² independently represents an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom;
n is 1 or 2, provided that when n is 1, m is 0 or 1 and when n is 2, m is 0, wherein if m is 0, then * represents a chiral centre;
R³ is (CH₂)ₐE, wherein a is 0, 1 or 2 and E is a group selected from: 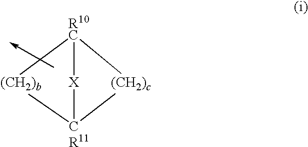
wherein:
X is O, S, NR¹², (CH₂), or a bond;
b is 1, 2, 3 or 4;
c is 1, 2 or 3;
v is 1 or 2;
R¹⁰ and R¹¹ are each independently H or C₁₋₄ alkyl; and
R¹² is H, C₁₋₆ alkyl, C(O)C₁₋₆ alkyl, SO₂—C₁₋₆ alkyl;
and wherein one or more pairs of hydrogen atoms on adjacent carbon or nitrogen atoms may be replaced by a corresponding number of double bonds, provided the ring system is not aromatic;
(ii) a carbocyclic spiro group containing 6 to 12 carbon atoms; 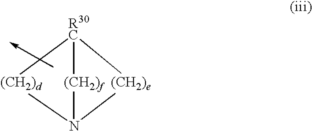
wherein:
d is 1, 2, 3 or 4;
e is 1, 2 or 3;
f is 1 or 2; and
R³⁰ is H or C₁₋₄ alkyl;
and wherein one or more pairs of hydrogen atoms on adjacent carbon atoms may be replaced by a corresponding number of double bonds, provided the ring system is not aromatic; 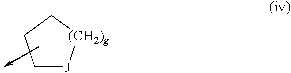
wherein:
g is 0, 1, 2 or 3;
J is NR⁴⁰; and
R⁴⁰ is C(O)C₁₋₆ alkyl, SO₂—C₁₋₆ alkyl; 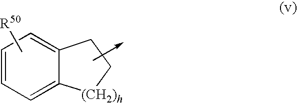
wherein:
h is 0, 1, 2 or 3; and
R⁵⁰ is H, C₁₋₈alkyl, C₁₋₈alkoxy, OH, halo, CF₃, OCF₃, SCF₃, hydroxy-C₁₋₆alkyl, C₁₋₄alkoxy-C₁₋₆alkyl and C₁₋₄alkyl-S—C₁₋₄alkyl;
(vi) —CH(cyclopropane)₂;
(vii) C₁₋₆alkyl, substituted by at least one substituent independently selected from C₁₋₆alkoxy, OH, halo, CF₃, OCF₃, SCF₃, CN, CF₂CF₃, CF₂—C₁₋₄alkyl, hydroxy-C₁₋₆alkyl, C₁₋₄alkoxy-C₁₋₆alkyl and C₁₋₄alkyl-S—C₁₋₄alkyl; and
(viii) C₃₋₈cycloalkyl-C₁₋₁₆alkyl; wherein the C₁₋₁₆alkyl moiety is substituted at any point other than at the junction with the C₃₋₈cycloalkyl moiety, by at least one substituent independently selected from C₁₋₆alkoxy, OH, halo, CF₃, OCF₃, SCF₃, CN, CF₂CF₃, CF₂—C₁₋₄alkyl, hydroxy-C₁₋₆alkyl, C₁₋₄alkoxy-C₁₋₆alkyl and C₁₋₄alkyl-S—C₁₋₄alkyl; and the C₃₋₈cycloalkyl moiety is optionally substituted by at least one substituent independently selected from C₁₋₈alkyl, C₁₋₆alkoxy, OH, halo, CF₃, OCF₃, SCF₃, CN, CF₂CF₃, CF₂—C₁₋₄alkyl, hydroxy-C₁₋₆alkyl, C₁₋₄alkoxy-C₁₋₆alkyl and C₁₋₄alkyl-S—C₁₋₄alkyl.

25. A compound according to claim 24, wherein R¹ is H.

26. A compound according to claim 24, wherein m is 0 and * represents the R or S enantiomer.

27. A compound according to claim 26, wherein * represents the S enantiomer.

28. A compound according to claim 23, wherein R² is aryl¹ optionally substituted by at least one substituent independently selected from B.

29. A compound according to claim 28, wherein B represents C₁₋₈alkyl, C₁₋₈alkoxy, aryl², Oaryl², SC₁₋₆alkyl, Saryl², C₃₋₆cycloalkyl, halo, CF₃, OCF₃, OCHF₂, CF₂CH₃, C₃₋₆cycloalkyl-C₁₋₄alkyloxy, and aryl²-C₁₋₄alkyl, wherein the aryl² group is independently optionally substituted by at least one group selected from C₁₋₆alkyl, C₃₋₆cycloalkyl, C₁₋₆alkoxy, OC₃₋₆cycloalkyl, halo, CN, OH, CF₃, CHF₂, OCF₃, OCHF₂, hydroxyC₁₋₆alkyl, C₁₋₄alkoxy-C₁₋₄alkyl, SC₁₋₆alkyl and SCF₃.

30. A compound according to claim 24, wherein R¹ is selected from (i), (ii), (vi), and (vii), as defined in claim 24.

31. A compound according to claim 24, selected from:
N-bicyclo[2.2.1]hept-2-yl-4-chloro-2-methoxy-N-[(3S)-pyrrolidin-3-yl]benzamide,
N-bicyclo[2.2.1]hept-2-yl-2-chloro-N-[(3S)-pyrrolidin-3-yl]benzamide,
N-[(1R,5S)-bicyclo[3.2.0]hept-3-yl]-2,3-dichloro-N-[(3S)-pyrrolidin-3-yl]benzamide,
2,3-dichloro-N-(dicyclopropylmethyl)-N-[(3S)-pyrrolidin-3-yl]benzamide,
2,3-dichloro-N-[(3S)-pyrrolidin-3-yl]-N-(3,3,3-trifluoropropyl)benzamide,
N-bicyclo[2.2.1]hept-2-yl-N-[(3S)-pyrrolidin-3-yl]-2-(trifluoromethyl)benzamide,
2-phenoxy-N-[(3S)-pyrrolidin-3-yl]-N-(3,3,3-trifluoropropyl)benzamide,
2-phenoxy-N-[(3S)-pyrrolidin-3-yl]-N-(2,2,2-trifluoroethyl)benzamide,
N-(2,2-difluoropropyl)-2-phenoxy-N-[(3S)-pyrrolidin-3-yl]benzamide,
N-(2,2-difluoroethyl)-2-phenoxy-N-[(3S)-pyrrolidin-3-yl]benzamide,
N-(2-methoxyethyl)-2-phenoxy-N-[(3S)-pyrrolidin-3-yl]benzamide,
2,4-dichloro-N-[(3S)-pyrrolidin-3-yl]-N-(3,3,3-trifluoropropyl)benzamide,
3,4-dichloro-N-[(3S)-pyrrolidin-3-yl]-N-(3,3,3-trifluoropropyl)benzamide,
2,3-dichloro-N-[(3S)-pyrrolidin-3-yl]-N-(2,2,2-trifluoroethyl)benzamide,
2-(ethylthio)-N-[(3S)-pyrrolidin-3-yl]-N-(3O,3)-trifluoropropyl)benzamide, and pharmaceutically and/or veterinarily acceptable derivatives thereof.

32. A pharmaceutical composition comprising a compound as claimed in claim 24 and a pharmaceutically acceptable adjuvant, diluent or carrier.

33. A method of treatment of a disorder in which the regulation of monoamine transporter function is implicated which comprises administering a therapeutically effective amount of a compound according to claim 24 to a patient in need of such treatment.

34. A method of treatment of a disorder in which the regulation of serotonin or noradrenaline is implicated which comprises administering a therapeutically effective amount of a compound according to claim 24 to a patient in need of such treatment.

35. A method according to claim 34, wherein the regulation of serotonin and noradrenaline is implicated.

36. A method of treatment of urinary disorders, depression, pain, premature ejaculation, hot flashes, ADHD or fibromyalgia, which comprises administering a therapeutically effective amount of a compound according to claim 24 to a patient in need of such treatment.

37. A method according to claim 36, wherein the urinary disorder is urinary incontinence, such as GSI or USI.

38. A method according to claim 36, wherein pain is treated.

39. A process for preparing a compound according to claim 24 comprising reacting a compound of formula X: 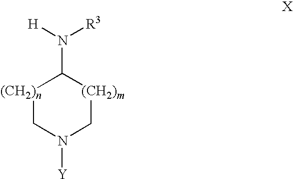
wherein R³, n and m are as defined in claim 24 and Y is R¹ or a protecting group, with an acid or acyl halide: R²COX, wherein X is OH or halo, and deprotecting if necessary or desired.
